(19) United States
(12) Patent Application Publication
(10) Pub. No.: US 2026/0137645 A1
(43) Pub. Date: May 21, 2026

(54) HIGHLY PURIFIED (R)-3’-HYDROXYBUTYL (R)-3’-HYDROXYBUTYRATE AND (R)-3-HYDROXYBUTYRATE AND COMBINATIONS WITH THERAPEUTICS

(71) Applicant: Ketone Catalyst Innovations, LLC, Ponte Vedra Beach, FL (US)

(72) Inventor: Anthony Capasso, Ponte Vedra Beach, FL (US)

(73) Assignee: Ketone Catalyst Innovations, LLC, Ponte Vedra Beach, FL (US)

(21) Appl. No.: 19/379,505

(22) Filed: Nov. 4, 2025

Related U.S. Application Data
(60) Provisional application No. 63/722,560, filed on Nov. 19, 2024.

(51) Int. Cl. A61K 31/22; A61K 31/05; A61K 31/12; A61K 31/202; A61K 31/352; A61K 36/05; A61K 36/28; A61K 36/324; A61K 36/87; A61K 36/9066; A61K 36/9068

(52) U.S. Cl. CPC A61K 31/22; A61K 31/05; A61K 31/12; A61K 31/202; A61K 31/352; A61K 36/05; A61K 36/28; A61K 36/324; A61K 36/87; A61K 36/9066; A61K 36/9068

(57) ABSTRACT

The present disclosure relates to biomedical and nutritional uses of highly purified (R)-3′-hydroxybutyl (R)-3′-hydroxybutyrate and (R)-3-hydroxybutyrate. In some embodiments, these compounds are employed for enhancing bodily functions and treating various disease states, including cognitive function, muscle power output, inflammation reduction, metabolic disorders, and mitochondrial function. The methods involve combining these ketone esters with natural supplements or medications to improve health outcomes. The disclosure further includes processes for manufacturing and purifying these compounds to ensure high specificity and efficacy in therapeutic applications, providing a comprehensive and enhanced strategy for advancing health and treating diseases.

### CROSS-REFERENCE

This application claims the benefit of, and priority to, U.S. Provisional Application No. 63/722,560, filed Nov. 19, 2024, and entitled HIGHLY PURIFIED (R)-3′-HYDROXYBUTYL (R)-3′-HYDROXYBUTYRATE AND (R)-3-HYDROXYBUTYRATE AND COMBINATIONS WITH THERAPEUTICS, the entire disclosure of which is incorporated herein by reference.

### FIELD OF THE INVENTION

The present disclosure relates to biomedical and nutritional uses of enantiomerically purified ketone bodies and ketone esters, particularly (R)-3′-hydroxybutyl (R)-3′-hydroxybutyrate and(R)-3-hydroxybutyrate, for enhancing bodily function and treating disease states such as in relationship to cognitive function, muscle power output, inflammation reduction, metabolic disorders, and mitochondrial function.

### BACKGROUND

The background related to production of (R)-3-hydroxybutyrate (BHB—herein) related compounds and associated uses may be summarized in the following patent applications, the contents of each of which are incorporated herein by reference: Process for Producing (R)-3-hydroxybutyl (R)-3-hydroxybutyrate, K Clarke, R L Veech & M T King, WO2014/140308 A1; Ketone Body and Ketone Body Ester for Reducing Muscle Breakdown, K Clarke & P Cox, WO2015/018913 A1; Process for Producing (R)-3-hydroxybutyl (R)-3-hydroxybutyrate, K Clarke, R L Veech & M T King, WO2014/140308 A1; Preparation of (3R)-hydroxybutyl-(3R)-hydroxybutyrate by Enzymatic Enantioselective Reduction, K Clarke, J Robertson & R L Veech, WO2010/120300 A1; and Compounds for Use in Reducing Liver Fat, K Clarke, WO2019/002828A1.

### SUMMARY

The disclosed methods leverage highly purified (R)-3′-hydroxybutyl (R)-3′-hydroxybutyrate for the unique biochemical properties of metabolically generated BHB to modulate metabolic pathways, gene expression, and cellular signaling, thereby offering a comprehensive approach to improving health and treating diseases such as metabolic disorders, neurodegenerative diseases, cardiovascular conditions, aging, sarcopenia, arthritis, mental disorders, and inflammatory diseases. Similar uses employing (R)-3-hydroxybutyrate (BHB) for ingestion also are disclosed.

In some embodiments, BHB may be sourced to the body through ingestion of highly purified (R)-3′-hydroxybutyl (R)-3′-hydroxybutyrate (also referred to herein as R,R BHB-BDO ester). As discussed in more detail in following sections, ingestion of R,R BHB-BDO ester may be processed by various processes in the body to generate R-BHB and R-BDO. Subsequently R-BDO may be metabolically processed to also yield R-BHB. Accordingly, methods to produce isomerically pure R,R BHB-BDO esters are important to afford highly pure R-BHB for clinical/medicinal purposes.

Enantiomerically purified ketone bodies and ketone esters ingested for biomedical and nutritional uses may be provided for administration to a human subject in amount and frequency conducive to enabling the beneficial biological response. In particular, in some embodiments, highly purified (R)-3′-hydroxybutyl (R)-3′-hydroxybutyrate may be provided for enhancing bodily function and treating various disease states. The methods described herein provide for production of a pure form of (R)-3′-hydroxybutyl (R)-3′-hydroxybutyrate via processes external to the human body.

In some methods, steps may include administering effective amounts of highly purified (R)-3′-hydroxybutyl (R)-3′-hydroxybutyrate to human subjects to achieve a range of therapeutic benefits, including but not limited to one or more of: inhibiting inflammasome activation, reducing pro-inflammatory cytokines, improving insulin sensitivity, enhancing mitochondrial biogenesis, and promoting muscle protein synthesis. Additionally, the present disclosure provides for highly purified (R)-3′-hydroxybutyl (R)-3′-hydroxybutyrate administration in an amount and frequency conducive to one or more of: improving cognitive function, reducing oxidative stress, enhancing cardiovascular health, and supporting neuroprotection. The disclosure further includes methods for manufacturing and purifying (R)-3′-hydroxybutyl (R)-3′-hydroxybutyrate to ensure high specificity and efficacy in therapeutic applications.

As mentioned, when ingested (R)-3′-hydroxybutyl (R)-3′-hydroxybutyrate may be metabolized in various manners to yield R-BHB in a user's blood stream. Herein, the acronym BHB will be reserved to mean the same as R-BHB, wherefore compositions including the S-BHB enantiomer will include the indication of the S prefix. R-BHB is a versatile and helpful energy molecule that supports metabolic processes across various animal species, especially during times when glucose is not available or is in limited supply.

In humans, the stereoisomer (R) β-hydroxybutyrate, which may also be called D β-hydroxybutyrate and herein BHB, is synthesized in the liver from fatty acids, β-hydroxy β-methylbutyrate, and ketogenic amino acids, primarily during periods of low carbohydrate intake. In some embodiments, this process may start with the production of acetoacetate, which may then be converted into BHB by the enzyme β-hydroxybutyrate dehydrogenase. BHB may also be produced through an alternative pathway involving butyrate metabolism, where it undergoes several conversions before being formed.

During ketosis, the concentration of BHB in the blood increases, providing an essential energy source for the brain and muscles when glucose is scarce. The physiological effects of BHB ingestion include reduced appetite, increased endurance, and improved mental clarity, though responses may vary based on individual factors.

Ingested (R)-3′-hydroxybutyl (R)-3′-hydroxybutyrate supplements may be metabolized in the gastrointestinal tract and subsequently may be rapidly absorbed into the bloodstream, with generated BHB crossing the blood-brain barrier to sustain cognitive function and promote energy efficiency by shifting the body's fuel use from glucose to ketones and fatty acids.

In some embodiments, the generated BHB may influence metabolic signaling pathways, inhibiting histone deacetylases (HDACs) to modulate gene expression, potentially offering anti-inflammatory and antioxidant benefits. Additionally, BHB may enhance mitochondrial function and stimulate the production of proteins linked to stress resistance, longevity, and overall metabolic health.

Despite its benefits, some methods of providing BHB supplementation may cause side effects such as gastrointestinal discomfort, electrolyte imbalances, and potential kidney stress, especially with high doses or prolonged use. As described herein, methods for highly purified and isomerically pure (R)-3′-hydroxybutyl (R)-3′-hydroxybutyrate are utilized for improved effectiveness and lowered side effect profiles. In more standard manufactured formulations for BHB supplementation, the various materials including examples such as 3-hydroxybutyl 3-hydroxybutyrate, may typically contain a mixture of the (R) and (S) enantiomers and in the case of the esters mixtures of (R)(R), (R)(S), (S)(R), and (S)(S) stereoisomers/enantiomers.

Referring now to FIGS. 1A-1J, stereochemical structures for the various isomers of potential relevance are illustrated. In FIG. 1A the focal moiety of the preset disclosure (R)-3′-hydroxybutyl (R)-3′-hydroxybutyrate 100 with the first (R) form chiral center 101 and the second (R) form chiral center 102 is illustrated. When this moiety is processed in the body of a user the initial products may include the structures illustrated in FIGS. 1B through 1D. Referring to FIG. 1B, BHB or (R)-3-hydroxybutyric acid 110 is illustrated with its (R) form chiral center 111 depicted. In the various environments within the cells and body structures of the user BHB may exist as the conjugate base (R)-3-hydroxybutyrate 120, which is illustrated with its (R) form chiral center 121. Proceeding to FIG. 1D, an initial metabolic product from (R)-3′-hydroxybutyl (R)-3′-hydroxybutyrate ingestion may also include (R)-1,3-butanediol 130. Its (R) form chiral center 131 is also illustrated.

In some embodiments, the (S) enantiomer (S) β-hydroxybutyrate or its ester isomers (S)(R), (R)(S) and (S)(S) 3-hydroxybutyl 3-hydroxybutyrate, may be a significant cause of some of the previously mentioned side effects. Proceeding to FIG. 1E, the (R)-3′-hydroxybutyl (S)-3′-hydroxybutyrate 140 stereoisomer is illustrated along with its chiral centers 141 and 142. Proceeding to FIG. 1F, the (S)-3′-hydroxybutyl (R)-3′-hydroxybutyrate 150 stereoisomer is illustrated along with its chiral centers 151 and 152. FIG. 1G, the (S)-3′-hydroxybutyl (S)-3′-hydroxybutyrate 160 stereoisomer is illustrated along with its chiral centers 161 and 162.

When any of these (S) form containing isomers (140,150 and 160) are metabolized the products may include (S)-3-hydroxybutyric acid 170 illustrated in FIG. 1H along with its chiral center 171. Similarly to the (R) form hydroxybutyric acid, the (S) form may exist as its conjugate base as illustrated in FIG. 1I, (S)-3-hydroxybutyrate 180 along with its chiral center 181. Similarly, as well the (S) form (S)-1,3-butanediol 190 is illustrated in FIG. 1J along with its chiral center 191. These various (S) form compounds may have a role in side effect profiles as mentioned previously.

Accordingly, the present disclosure describes methods of production of (R)-3′-hydroxybutyl (R)-3′-hydroxybutyrate supplies in highly purified forms. There are also descriptions of methods of purifying 3′-hydroxybutyl-3′-hydroxybutyrate supplies to generate enantiomerically pure forms, whether (R)(R) or (S)(S) forms. Moreover, In some embodiments herein, mixtures of (R)-3′-hydroxybutyl (R)-3′-hydroxybutyrate may be formed with other additives to ameliorate noted side effects.

Current processing alternatives for the production (R)-3-Hydroxybutyl (R)-3-Hydroxybutyrate result in formulations that are at best 80-85% pure for the desired enantiomer. Accordingly, there are numerous other isomers that may reduce the effectiveness and/or generate side effect profiles in standard processing. A key aspect of the instant application is to form highly purified (R)-3-Hydroxybutyl (R)-3-Hydroxybutyrate for its improvement in these respects. In some embodiments, highly purified (R)-3-Hydroxybutyl (R)-3-Hydroxybutyrate samples may be at least 95% pure. In some other examples, highly purified (R)-3-Hydroxybutyl (R)-3-Hydroxybutyrate samples may be at least 99% pure.

In some embodiments, a method for manufacturing a purified (R)-3′-hydroxybutyl (R)-3′-hydroxybutyrate may involve various techniques to ensure high specificity and efficiency in removing the undesired S-form stereoisomers including (R)(S), (S)(R), and (S)(S) stereoisomers/enantiomers. One approach, for example, may use an enzyme, possibly derived from a microbial source, that selectively targets and eliminates the S-form. In some other examples, another method may combine enzymatic steps with high-performance liquid chromatography (HPLC) for further purification. Additionally, a continuous flow reactor system with immobilized enzymes may be employed for scalable production, or a batch processing method may be used for small-scale or high-purity production. In some approaches, additional focus on the stereo-purity of starting materials is included. In still further approaches, careful optimization of process variables for processing steps may result in improved purity.

Isolates which have been enantiomerically purified may be formulated into various products that may be used to enhance bodily function or treat disease states, as described in detail in further sections of the present disclosure. In some embodiments, mixtures may be formulated using the purified (R)-3′-hydroxybutyl (R)-3′-hydroxybutyrate enantiomer. The mixtures may include compositions formed by adding controlled amounts of the (R)(S), (S)(R), and (S)(S) stereoisomers/enantiomers variously.

In some other examples, enantiomerically purified (R)-3′-hydroxybutyl (R)-3′-hydroxybutyrate may be mixed with other substances, as described in later sections, to reduce or modify the side effects of 3-hydroxybutyl-3-hydroxybutyrate ingestion as mentioned previously, including additions which may improve the palatability of the formulation. However, In some embodiments the purified isolate alone may be utilized.

According to one aspect of the present disclosure, a method for inhibiting NOD-like receptor family pyrin domain containing 3(NLRP 3 ) inflammasome activation in a subject may include administering an effective amount of the highly purified (R)-3′-hydroxybutyl (R)-3′-hydroxybutyrate to the subject. According to another aspect, the method may include examples wherein the inhibition of NLRP3 inflammasome activation reduces the secretion of pro-inflammatory cytokines IL-1β and IL-18. Furthermore, according to yet another aspect, a method may include examples for reducing IL-6 levels in a subject includes administering an effective amount of (R)-3′-hydroxybutyl (R)-3′-hydroxybutyrate to the subject, wherein the result of metabolism, BHB may activate the GPR109A receptor and inhibit NF-kB activity. According to another aspect, the method may include examples wherein the reduction of IL-6 levels is achieved through the antioxidative effects of BHB.

In some other examples, a method for inhibiting Tumor Necrosis Factor Alpha (TNF-alpha) production in a subject may include administering an effective amount of highly purified (R)-3′-hydroxybutyl (R)-3′-hydroxybutyrate to the subject, wherein generated BHB activates the GPR109A receptor and nuclear factor erythroid 2-related factor 2 (Nrf 2) pathway. According to another aspect, the method may include examples wherein the inhibition of TNF-alpha production is achieved through the antioxidative effects of BHB.

According to yet another aspect, a method for increasing adenosine levels in a subject may include administering an effective amount of highly purified (R)-3′-hydroxybutyl (R)-3′-hydroxybutyrate to the subject, wherein the increased adenosine levels may exert anti-inflammatory effects via A1 and A2 receptors. According to another aspect, the method may include examples wherein the anti-inflammatory effects may include the reduction of IL-6 and TNF-alpha levels and the increase of IL-10 levels.

In some other examples, a method for promoting the polarization of macrophages towards the M2 phenotype in a subject includes administering an effective amount of highly purified (R)-3′-hydroxybutyl (R)-3′-hydroxybutyrate to the subject. In these examples, the BHB may inhibit histone deacetylases (HDACs) and activate the G-protein coupled receptor 109A (GPR109A) receptor. According to another aspect, the method may include examples wherein the M2 macrophage phenotype is associated with anti-inflammatory properties.

According to yet another aspect, a method for activating the mechanistic Target of Rapamycin Complex 1 (mTORC1) signaling pathway in a subject may include administering an effective amount of highly purified (R)-3′-hydroxybutyl (R)-3′-hydroxybutyrate to the subject, wherein the activation of mTORC1 promotes muscle protein synthesis. According to another aspect, the method may include examples wherein the activation of mTORC1 is indicated by the phosphorylation of p70S6 kinase (p70S6K) and eukaryotic translation initiation factor 4E-binding protein 1 (4E-BP1).

According to yet another aspect, a method for reducing systemic inflammation in a subject may include administering an effective amount of highly purified (R)-3′-hydroxybutyl (R)-3′-hydroxybutyrate to the subject, wherein BHB inhibits the NF-κB signaling pathway. According to another example, the method may include reduction of systemic inflammation that may occur in muscle wasting due to their association with chronic inflammation.

In some embodiments, a method for mitigating oxidative stress in a subject may include administering an effective amount of highly purified (R)-3′-hydroxybutyl (R)-3′-hydroxybutyrate to the subject, wherein BHB enhances the activity of antioxidant enzymes and reduces the production of reactive oxygen species (ROS). In some of these examples, the method may include examples wherein the mitigation of oxidative stress may be achieved through the activation of the Nrf2 pathway.

According to yet another aspect, a method for improving glucose management in a subject may include administering an effective amount of highly purified (R)-3′-hydroxybutyl (R)-3′-hydroxybutyrate to the subject, wherein BHB resulting from metabolism improves glucose control and reduces fructosamine levels. According to another aspect, the method may include examples wherein the improved glucose management is indicated by lower levels of Hemoglobin A1c (HbA1c) and Homeostasis Model Assessment of Insulin Resistance (HOMA-IR). In some embodiments, highly purified (R)-3′-hydroxybutyl (R)-3′-hydroxybutyrate may be combined with other pharmaceuticals in the formulation such as in non-limiting examples, metformin, diuretics and glucagon like protein-1 receptor agonists include but not limited to semaglutide, liraglutide, exenatide, dulaglutide, lixisenatide, albiglutide and efpeglenatide. In some other examples, BHB may be combined with dual glucagon like protein-1 receptor agonist and glucose-dependent insulinotropic polypeptide receptor agonist molecules such as tirzepatide.

According to yet another aspect, a method for protecting a subject against neurodegeneration may include administering an effective amount of highly purified (R)-3′-hydroxybutyl (R)-3′-hydroxybutyrate to the subject. In these examples, the metabolically produced BHB may serve as an alternative energy source for neurons. In some of these examples, the method may include cases wherein the protection against neurodegeneration is achieved through the reduction of oxidative stress, inhibition of neuroinflammation, enhancement of mitochondrial function, stimulation of autophagy, and regulation of apoptotic pathways.

In some embodiments, a method for enhancing cognitive function in a subject may include administering an effective amount of highly purified (R)-3′-hydroxybutyl (R)-3′-hydroxybutyrate to the subject.

According to another aspect, a method for improving cognitive performance in subjects with psychiatric conditions includes administering highly purified (R)-3′-hydroxybutyl (R)-3′-hydroxybutyrate and conducting EEG tests to measure brain activity. According to yet another aspect, a method for analyzing the purity of a pharmaceutical product includes performing stereoisomer analysis to identify and quantify impurities.

According to another aspect, a method for treating metabolic disorders in a subject includes administering a combination of highly purified (R)-3′-hydroxybutyl (R)-3′-hydroxybutyrate and an anti-diabetic drug to the subject.

According to yet another aspect, a method for enhancing mitochondrial function in a subject includes administering a composition containing a purified R-form enantiomer of a ketone ester and measuring mitochondrial biogenesis markers.

According to yet another aspect, a method for treating cardiovascular conditions includes administering highly purified (R)-3′-hydroxybutyl (R)-3′-hydroxybutyrate to a subject. In some of these examples, the method may include monitoring cardiovascular markers. In some embodiments, the highly purified (R)-3′-hydroxybutyl (R)-3′-hydroxybutyrate composition may include more than 90% composition of the highly purified (R)-3′-hydroxybutyl (R)-3′-hydroxybutyrate enantiomer. In some embodiments, the highly purified (R)-3′-hydroxybutyl (R)-3′-hydroxybutyrate composition may include more than 99% composition of the highly purified (R)-3′-hydroxybutyl (R)-3′-hydroxybutyrate.

According to some aspects methods for enhancing various aspects of health and wellness through the administration of highly purified (R)-3′-hydroxybutyl (R)-3′-hydroxybutyrate or (R)-3-hydroxybutyrate in combination with specific supplements or pharmaceuticals are described. These combinations may target a wide range of physiological and cognitive functions, aiming to improve overall health outcomes.

Several embodiments may address cognitive enhancement, including methods for improving cognitive function, focus, and clarity. These embodiments may involve combinations of highly purified (R)-3′-hydroxybutyl (R)-3′-hydroxybutyrate or (R)-3-hydroxybutyrate with supplements like caffeine, L-theanine, and acetyl-L-carnitine, which may be known for their cognitive benefits. The combinations may support mental performance and resilience, particularly in stressful environments.

Other embodiments focus on mood and emotional well-being. Some of these methods may relate to reducing anxiety, stress, and symptoms of depression. Combinations of highly purified (R)-3′-hydroxybutyl (R)-3′-hydroxybutyrate or (R)-3-hydroxybutyrate with supplements such as 5-HTP, ashwagandha, and SAM-e define embodiments for their potential to enhance mood and emotional stability, providing a holistic approach to mental health.

In some embodiments, improvements related to physical health may include methods for enhancing muscle recovery, strength, and endurance. These involve combinations of highly purified (R)-3′-hydroxybutyl (R)-3′-hydroxybutyrate or (R)-3-hydroxybutyrate with branched-chain amino acids (BCAAs), creatine, and beetroot extract, aiming to support athletic performance and recovery from physical exertion.

Metabolic health may be another area, with embodiments focusing on improving insulin sensitivity, metabolic rate, and flexibility. Combinations with metformin, berberine, and chromium picolinate may enhance metabolic processes and support weight management as non-limiting examples.

Several embodiments may address cardiovascular and immune health, with methods for supporting heart function and enhancing immune resilience. Combinations of highly purified (R)-3′-hydroxybutyl (R)-3′-hydroxybutyrate or (R)-3-hydroxybutyrate with omega-3 fatty acids, grape seed extract, and echinacea may promote cardiovascular health and bolster the immune system.

Some embodiments may also target specific health conditions, such as reducing symptoms of arthritis, fibromyalgia, and eczema. In some embodiments combinations of highly purified (R)-3′-hydroxybutyl (R)-3′-hydroxybutyrate or (R)-3-hydroxybutyrate with turmeric, SAM-e, and primrose oil as non-limiting examples, may alleviate symptoms and improve quality of life for individuals with these conditions.

Skin, hair, and joint health may be addressed by various examples through combinations of highly purified (R)-3′-hydroxybutyl (R)-3′-hydroxybutyrate or (R)-3-hydroxybutyrate with collagen peptides, biotin, and glucosamine. These claims focus on enhancing the structural integrity and appearance of skin and hair, as well as supporting joint flexibility and health.

Some examples may relate to detoxification and liver health involving combination of highly purified (R)-3′-hydroxybutyl (R)-3′-hydroxybutyrate or (R)-3-hydroxybutyrate with milk thistle extract, chlorella, and dandelion root as non-limiting examples. These methods may focus on supporting the body's natural detoxification processes and promoting liver function.

### BRIEF DESCRIPTION OF DRAWINGS

FIGS. 1A-1J.—illustrate the various enantiomers of 3-hydroxybutyrate, 1,3-butanediol, and the enantiomers/stereoisomers of 3′-hydroxybutyl-3′-hydroxybutyrate.

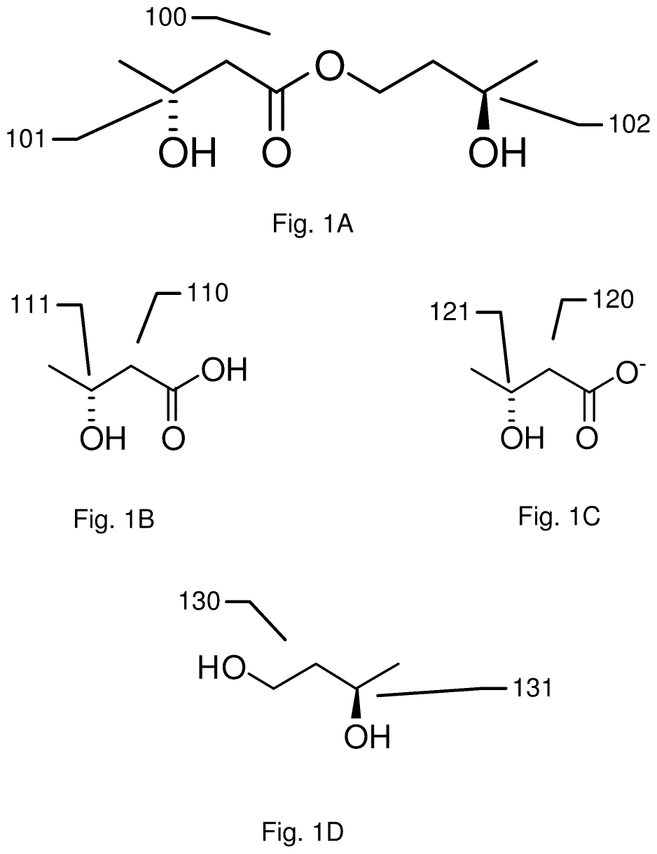

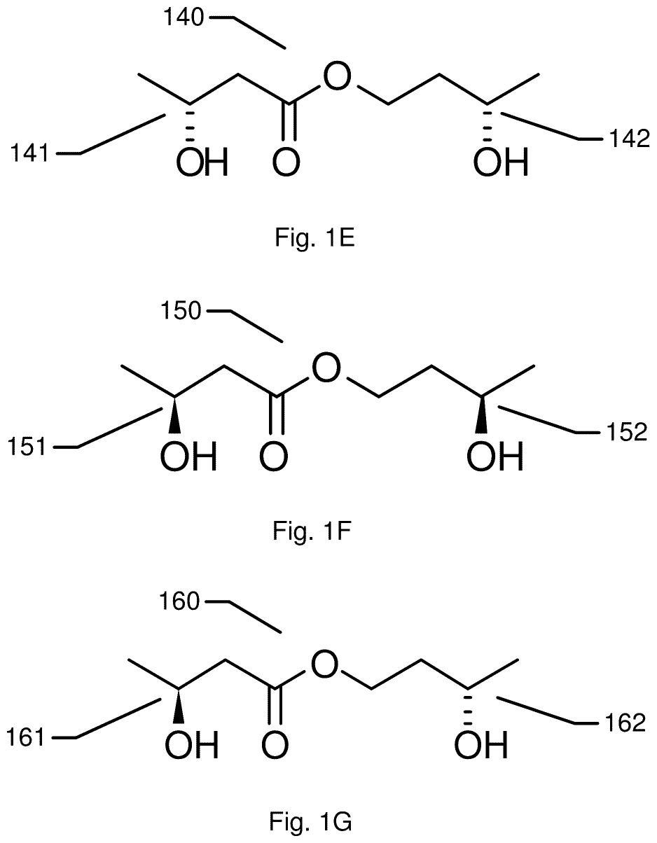

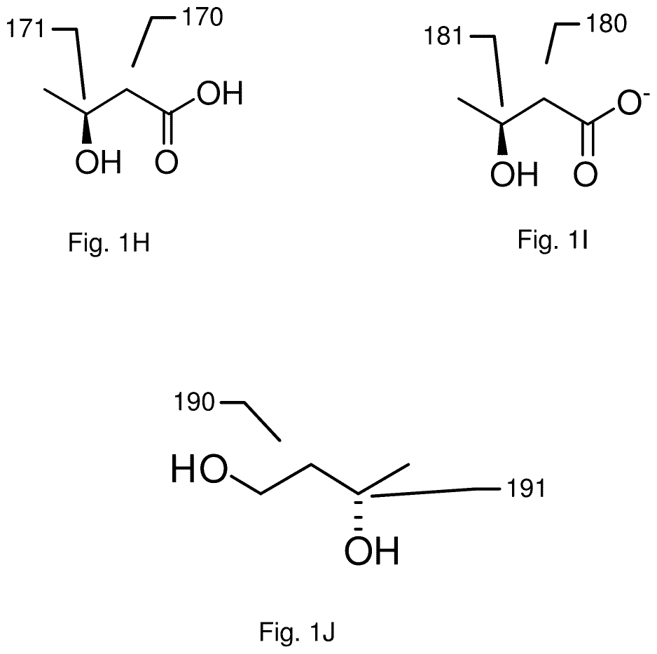

### DETAILED DESCRIPTION

According to the present disclosure, highly purified (R)-3′-hydroxybutyl (R)-3′-hydroxybutyrate compositions are described. The metabolism of ingested highly purified (R)-3′-hydroxybutyl (R)-3′-hydroxybutyrate results in the chemical moiety variously termed D-β-hydroxybutyrate, (R)-beta-hydroxybutyrate (BHB) and (R)-3-hydroxybutyric acid, Accordingly, BHB may be provided in a highly pure form from highly purified (R)-3′-hydroxybutyl (R)-3′-hydroxybutyrate. This BHB result may be essentially bioidentical to BHB synthesized in the liver through the metabolism of fatty acids, β-hydroxy β-methylbutyrate, and ketogenic amino acids. In humans the conversion process may include the conversion of these compounds into acetoacetate, a ketone body produced during fasting. The enzyme β-hydroxybutyrate dehydrogenase may catalyze the conversion of acetoacetate into D-β-hydroxybutyrate, which plays an important role in energy metabolism during periods of low carbohydrate availability.

Additionally, a form of D-β-hydroxybutyrate is provided through an alternative metabolic pathway that does not involve acetoacetate. In this pathway, butyrate may be metabolized into butyryl-CoA, and converted into crotonyl-CoA, followed by β-hydroxybutyryl-CoA, poly-β-hydroxybutyrate, and finally D-β-(D-β-hydroxybutyryloxy)-butyrate. The enzyme hydroxybutyrate-dimer hydrolase catalyzes the final step, converting D-β-(D-β-hydroxybutyryloxy)-butyrate into D-β-hydroxybutyrate.

The BHB provided through metabolism of highly purified (R)-3′-hydroxybutyl (R)-3′-hydroxybutyrate is essentially bioidentical with a concentration of BHB in human blood plasma that increases during ketosis, a metabolic state where the body shifts to utilizing fat as its primary energy source due to low glucose levels. The present disclosure provides for an elevation in BHB via an alternative source than its natural formation. According to the present disclosure, BHB is metabolized from highly purified (R)-3′-hydroxybutyl (R)-3′-hydroxybutyrate and may be provided in a pure form as an alternative energy source for the brain and skeletal muscles, particularly when glucose availability is limited.

According to the present disclosure, highly purified (R)-3′-hydroxybutyl (R)-3′-hydroxybutyrate is produced and formulated in various forms for ingestion or injection into a user. The physiological effects of highly purified (R)-3′-hydroxybutyl (R)-3′-hydroxybutyrate ingestion include reduced appetite, increased endurance, and improved mental clarity, though responses may vary based on individual factors. Ingested highly purified (R)-3′-hydroxybutyl (R)-3′-hydroxybutyrate supplements may be rapidly absorbed into the bloodstream, crossing the blood-brain barrier to sustain cognitive function and promote energy efficiency by shifting the body's fuel use from glucose to ketones and fatty acids.

When highly purified (R)-3′-hydroxybutyl (R)-3′-hydroxybutyrate BHB is ingested, it may be processed in the user's gastrointestinal (GI) tract to form both BHB and 1,3-butanediol. These species may be rapidly absorbed into the bloodstream, where the BHB may act as an alternative to glucose for energy and/or produce various therapeutic effects as mentioned herein. In an alternative mechanism, (R)-1,3 Butanediol may course through the blood system of the user and reach the liver where it may subsequently be converted to BHB, forming a longer-term presentation of BHB into the bloodstream following ingestion of highly purified (R)-3′-hydroxybutyl (R)-3′-hydroxybutyrate.

Once in the bloodstream, BHB crosses the blood-brain barrier, providing the brain with a stable energy source, which is especially important when carbohydrate intake is low. This facilitates the maintenance of cognitive function during periods of reduced glucose availability. The presence of BHB in the blood promotes a state of ketosis, where the body shifts from using glucose as its primary fuel to relying on ketones and fatty acids, optimizing energy utilization.

BHB may also play a role in regulating various metabolic signaling pathways. It has been shown that BHB may inhibit histone deacetylases (HDACs), which may modulate gene expression and potentially provide anti-inflammatory and antioxidant benefits. Additionally, BHB may enhance mitochondrial efficiency and promote the production of proteins associated with stress resistance, longevity, and overall metabolic health.

BHB inhibited HDAC3 and caused acetylation of H3K14 in the Claudin-5 promoter, thereby promoting claudin-5 generation and antagonizing diabetes-associated cardiac microvascular hyperpermeability. https://link.springer.com/article/10.1007/s00125-020-05305-2

The β-Hydroxybutyrate Receptor HCA2 Activates a Neuroprotective Subset of Macrophages. https://www.ncbi.nlm.nih.gov/pmc/articles/PMC4325597/

May induce oxidative stress resistance through the expression of FOXO3a and MT2 in mouse kidneys, thus improving the metabolic profile and redox status (https://www.frontiersin.org/journals/immunology/articles/10.3389/fimmu.2022.981285/full

Induction of Foxo3a appears to be a direct effect of HDAC inhibition, as HDAC1 and HDAC2 are found on its promoter, knockdown of both relieves HDAC-mediated Foxo3a repression, and BHB causes hyperacetylation of histones at the Foxo3a promoter that results in increased Foxo3a expression https://www.ncbi.nlm.nih.gov/pmc/articles/PMC6640868/

BHB regulates Bdnf (brain derived neurotrophic factor) expression in the mouse brain by a similar mechanism after exercise.

The physiological effects of (R)-3′-hydroxybutyl (R)-3′-hydroxybutyrate ingestion may include reduced appetite, increased endurance, and improved mental clarity. However, individual responses to (R)-3′-hydroxybutyl (R)-3′-hydroxybutyrate supplementation may vary depending on factors such as diet, physical activity, and metabolic rate.

Ingesting 3-hydroxybutyl-3-hydroxybutyrate, particularly through racemic exogenous supplements, may have several side effects, although most are generally mild. The side effects may vary depending on the individual's health status, dosage, and frequency of consumption. In some embodiments, formulations with highly enantiomerically pure (R)-3′-hydroxybutyl (R)-3′-hydroxybutyrate may minimize occurrence with side effects. In some other examples, mixtures of highly enantiomerically pure (R)-3′-hydroxybutyl (R)-3′-hydroxybutyrate and other compounds which alleviate the side effects when they occur may be formulated. Side effects may include gastrointestinal issues including nausea, diarrhea and stomach discomfort, electrolyte imbalance and possible kidney stress, hypoglycemia, bad breath, and elevated heart rates. In some embodiments, particularly with racemic mixtures of 3-hydroxybutyl-3-hydroxybutyrate, unpleasant taste may be a side effect. In some embodiments, forming combinatorial products of (R)-3′-hydroxybutyl (R)-3′-hydroxybutyrate with other materials may ameliorate these side effects.

### Highly Purified (R)-3′-hydroxybutyl (R)-3′-hydroxybutyrate and Product Manufacturing

In some embodiments, the method for manufacturing purified (R)-3′-hydroxybutyl (R)-3′-hydroxybutyrate may involve using a specific enzyme that selectively targets and removes the undesired S-form enantiomer from the starting materials. This enzyme may be derived from a microbial source known for enantioselective properties, ensuring high specificity and efficiency in the purification process. In another example, the method may utilize a combination of enzymatic and chromatographic techniques, where the initial enzymatic step may reduce the S-form enantiomer concentration of starting materials, followed by high-performance liquid chromatography (HPLC) to achieve further purification and ensure the starting materials are pure. In some embodiments an additional HPLC step may be utilized to ensure the final product meets the desired purity standards.

In some additional embodiments, a method may involve the use of a continuous flow reactor system, where the starting materials may be continuously fed into a reactor containing immobilized enzymes, allowing for a scalable and efficient purification process. This system may be optimized for various flow rates and reaction times to maximize yield and purity. Furthermore, an alternative example might employ a batch processing approach, where the starting materials may be mixed with the enzyme in a controlled environment. In some embodiments, the reaction may proceed to completion before the product is isolated and purified. This batch method may be particularly useful for small-scale production or for producing high-purity samples for research purposes. Each of these examples may demonstrate that the (R)-3′-hydroxybutyl (R)-3′-hydroxybutyrate may be produced efficiently and with high purity.

In some embodiments, a method of manufacturing a purified enantiomerically pure ester with purified BHB starting material may include mixing to a 1:1 mixture (+50%) of (purified, R)-1,3 Butanediol, and (purified R)-Ethyl 3 hydroxybutyrate and transesterification enzyme (1% to 25% by weight). The mixture may be incubated at 25° C. to 100° C. under reduced pressure (0 mm Hg to 759 mm Hg) for an appropriate period of time (as monitored by GC/MS). Upon completion of the reaction, an appropriate solvent may be added and the solids removed by filtration. Solvent and excess reagents may then be removed via distillation under reduced pressure. In some embodiments, wipe film evaporation (WFE) may be used as a highly efficient method used to separate compounds based on their volatility through evaporation. In some embodiments, the product result of the various reaction and purification method steps may be processed by WFE by spreading a thin film of a product over a heated surface, which allows for rapid evaporation of the volatile components, leaving behind the non-volatile residue. WFE may expose the product to less severe conditions such as to high temperatures for only seconds, and continuous operation may be facilitated. WFE may facilitate process optimization due to the quick analysis of process changes on dependent variables (like product purity).

### Highly Purified (R)-3′-hydroxybutyl (R)-3′-hydroxybutyrate and Purity Analysis

In some embodiments, a method for analyzing the purity of a (R)-3′-hydroxybutyl (R)-3′-hydroxybutyrate related composition may involve performing stereoisomer analysis using high-performance liquid chromatography (HPLC) equipped with a chiral column. This setup may allow for the separation and quantification of different enantiomers and more generally stereoisomers present in the sample, ensuring precise identification and measurement of impurities or to the control of different mixtures between stereoisomers, although refinements may be needed In some embodiments for the complexity of esters with dual chiral centers. In other examples, a high degree of purity of (R)-3′-hydroxybutyl (R)-3′-hydroxybutyrate samples may be controlled with the method.

In another example, chiral gas chromatography-mass spectrometry (GC-MS) is employed to analyze the stereoisomeric composition, providing high sensitivity and specificity in detecting even trace amounts of impurities.

Additionally, an example could utilize nuclear magnetic resonance (NMR) spectroscopy to determine the stereochemical configuration and purity of the product, offering detailed structural information.

For a more automated and high-throughput approach, an example might incorporate an automated liquid handling system coupled with HPLC or GC-MS, enabling rapid and consistent analysis of multiple samples. Furthermore, an example may involve the use of capillary electrophoresis (CE) with a chiral selector to achieve high-resolution separation of enantiomers, which may be particularly useful for complex mixtures. Each of these examples facilitates consistent adaptability and effectiveness across various analytical techniques, providing robust and reliable purity analysis of pharmaceutical products.

### Highly Purified (R)-3′-hydroxybutyl (R)-3′-hydroxybutyrate and Inflammation

Chronic inflammation and metabolic disorders present significant challenges in modern healthcare. These conditions often lead to a cascade of adverse health effects, including cardiovascular diseases, neurodegenerative disorders, and impaired cognitive function. Traditional treatments for these conditions typically involve anti-inflammatory drugs, lifestyle modifications, and various pharmacological interventions aimed at managing symptoms rather than addressing underlying causes. Despite these efforts, there remains a substantial need for more effective and targeted therapies that may mitigate inflammation and improve metabolic health without causing significant side effects.

The disclosed method may address chronic inflammation and metabolic disorders through the administration of highly purified forms of (R)-3′-hydroxybutyl (R)-3′-hydroxybutyrate or mixtures made with highly purified (R)-3′-hydroxybutyl (R)-3′-hydroxybutyrate. In some embodiments, administration of highly purified (R)-3′-hydroxybutyl (R)-3′-hydroxybutyrate containing therapeutics may leverage properties of BHB to inhibit NLRP3 inflammasome activation, reduce pro-inflammatory cytokines, and enhance mitochondrial function. By targeting multiple pathways involved in inflammation and metabolism, the method may offer a comprehensive solution that addresses the root causes of these conditions.

### NLRP3 Inflammasome

### VGLUT/K Channels—

VGLUT inhibition may be a mechanism by which BHB can reduce excitatory glutamate neurotransmission without affecting inhibitory GABA neurotransmission

NLRP3 inflammasome activation facilitates the maintenance and trafficking of haematopoietic stem cells and haematopoiesis.

https://www.frontiersin.org/journals/immunology/articles/10.3389/fimmu.2021.765199/full

In some embodiments, a composition containing highly purified (R)-3′-hydroxybutyl (R)-3′-hydroxybutyrate may be administered to a subject to reduce inflammation, with the composition being delivered orally in the form of a liquid solution. The subject's inflammatory markers in the blood may be monitored using standard blood tests, such as ELISA (enzyme-linked immunosorbent assay) to measure levels of cytokines like IL-6 and TNF-alpha.

In another example a highly purified (R)-3′-hydroxybutyl (R)-3′-hydroxybutyrate composition may be encapsulated in a slow-release capsule to provide a sustained release of the active ingredient over time, ensuring prolonged anti-inflammatory effects. The monitoring of inflammatory markers may be enhanced by using wearable biosensors that continuously measure biomarkers in sweat, providing real-time data on the subject's inflammatory status. In a further example, compositions containing highly enantiomerically purified (R)-3′-hydroxybutyl (R)-3′-hydroxybutyrate may be combined with an anti-inflammatory drug, such as ibuprofen, to provide a synergistic effect in reducing inflammation. The subject's response to the treatment may be monitored through blood tests and imaging techniques to assess inflammation in specific tissues.

In some embodiments, the method for reducing inflammation in a subject may involve administering a ketone ester composition that includes (R)-3-hydroxybutyrate. This composition may be delivered orally in the form of a liquid solution, a gel, or a solid such as a tablet or capsule. The administration may be performed in a single dose or multiple doses over a specified period, depending on the severity of the inflammation and the subject's response to the treatment. The highly purified (R)-3′-hydroxybutyl (R)-3′-hydroxybutyrate containing composition may be combined with other anti-inflammatory agents, such as omega-3 fatty acids or curcumin, bromelain, Boswellia, or Ginger, to enhance their anti-inflammatory effects.

There may be numerous ways that the highly purified (R)-3′-hydroxybutyl (R)-3′-hydroxybutyrate compositions may elicit a medically effective response through the metabolically produced BHB. In a first example, BHB may act to inhibit NLRP3 inflammasome production or activation. In some understandings, the NLRP3 inflammasome may function as an intracellular sensor that, when activated by a variety of pro-inflammatory stimuli, may trigger secretion of pro-inflammatory cytokines. In some embodiments, BHB may directly inhibit activation of the NLRP3 inflammasome, thereby preventing secretion of pro-inflammatory mediators, including Interleukins such as IL-1β and IL-18. In some other examples, BHB may also act indirectly to suppress NLRP3 activation by inhibiting K+ efflux from cells. In this way, this may inhibit an upstream signal for NLRP3 inflammasome activation.

There may be numerous disease states where such inhibition of NLRP3 inflammasome may be of clinical relevance. These disease states may include Type 2 diabetes mellitus (T2D), Rheumatoid arthritis (RA), Atherosclerosis, Alzheimer's disease (AD), Psoriatic arthritis, Ankylosing spondylitis, Gout, Myocardial infarction (MI), Stroke, Septic shock syndrome, and Acute Respiratory Distress Syndrome (ARDS) as a non-limiting set of examples. There may also be more rarely occurring disease states where such means of inhibition of inflammation provided by highly purified (R)-3′-hydroxybutyl (R)-3′-hydroxybutyrate containing compositions may be important and helpful including Cryopyrin-associated periodic syndrome (CAPS), Familial Mediterranean fever (FMF), Hemophagocytic lymphohistiocytosis (HLH), Hyperimmunoglobulinemia D and periodic fever syndrome (HIDS)/Mevalonate kinase deficiency (MKD), Tumor Necrosis Factor Receptor-1-associated syndrome (TRAPS), Pyogenic Arthritis, Pyoderma Gangrenosum, and Acne syndrome (PAPA) as a further non-limiting set of examples.

In some embodiments, highly purified (R)-3′-hydroxybutyl (R)-3′-hydroxybutyrate containing compositions may be effective through the action of BHB to inhibit NF-kB via GPR109A. In some embodiments, this may subsequently reduce IL-6. BHB may function as an activator of the GPR109A receptor, which is expressed in various cell types, including those involved in immune responses. Activation of the GPR109A receptor by R-BHB may support anti-inflammatory effects primarily through the suppression of NF-kappaB, a transcription factor regulating the expression of numerous pro-inflammatory cytokines.

In another simultaneous example, the promoter region of the IL-6 gene contains a binding site for NF-κB, which may be essential for IL-6 gene activation. This NF-kB may itself be sensitive to oxidative stress. The role of BHB may be to provide antioxidative effects on multiple levels such as regulation from decreasing reactive oxygen species by preventing reverse transport chain or increasing the expression of endogenous antioxidants such as SOD2 or MT2. The binding of NF-κB to this site facilitates the inducibility of the IL-6 promoter. By reducing NF-kappaB activity, BHB potentially decreases pro-inflammatory cytokine production. Additionally, BHB may also exert anti-inflammatory effects through other pathways, such as the inhibition of histone deacetylases (HDACs), which may indirectly affect NF-kappaB activity.

There may be numerous disease states where elevated levels of IL-6 are found and where modulation by treatment with highly purified (R)-3′-hydroxybutyl (R)-3′-hydroxybutyrate containing compositions to reduce levels of IL-6 may result in effective treatments. These disease states may include Type 2 Diabetes Mellitus, Cardiovascular Diseases (including myocardial infarction and atherosclerosis), Obesity, Asthma, Chronic Obstructive Pulmonary Disease (COPD), Rheumatoid Arthritis (RA), Alzheimer's Disease (AD), Inflammatory Bowel Disease (IBD) (including Crohn's Disease and Ulcerative Colitis) as a set of non-limiting examples. There may also be relevance to more rarely occurring disease states as well including Systemic Lupus Erythematosus (SLE), Severe Sepsis and Septic Shock, Multiple Sclerosis, HIV Infection, Systemic Juvenile Idiopathic Arthritis (sJIA), Castleman Disease (CD), and Chronic inflammatory pulmonary diseases (such as interstitial lung disease) as a set of non-limiting examples.

In some embodiments, BHB may inhibit TNF-alpha via NF-kB inhibition. Similarly to IL-6, TNF-alpha may also be activated by NF-kB so BHB may inhibit it via the GPR109A activation. Moreover, BHB may also activate Nrf2, which was shown to inhibit supress TNF-alpha production. Furthermore, the antioxidative effects may also lower TNF-alpha activity. There may be various disease states where lowered TNF-alpha levels may be of clinical relevance. In some embodiments, in the elderly, higher TNF-alpha levels were associated with mortality in men but not women. In some other non-limiting examples, diseases associated with high TNF-alpha levels may include Rheumatoid Arthritis, Psoriatic Arthritis, Juvenile Idiopathic Arthritis, Ankylosing Spondylitis, Psoriasis, Crohn's Disease, Ulcerative Colitis, COVID-19, Autoinflammatory Syndromes, Septic Shock, Inflammatory Bowel Disease (IBD), Autoimmune Lymphoproliferative Syndrome (ALPS).

In some embodiments, BHB may cause an adenosine-mediated anti-inflammatory effect. The mechanism involving adenosine may contribute to anti-inflammatory effects of BHB. In some embodiments, ketone metabolism may lead to an increase in adenosine levels. Adenosine may exert anti-inflammatory effects through its interaction with A1 and A2 receptor subtypes in both peripheral and central tissues. BHB may mitigate HIF-1α-induced inflammation and oxidative stress by activating the adenosine A1 receptor, thus offering a potential therapeutic pathway for managing inflammation.

The most common effects of adenosine receptor anti-inflammatory actions are lower levels of IL-6 and TNF-alpha. However, it may also activate A2A receptors and augment production of IL-10, which is an anti-inflammatory cytokine that blocks the expression of multiple pro-inflammatory cytokines, including IL-1, IL-6 and TNF-alpha and inhibiting the NF-kB pathway. Such action may have clinical relevance for disease states, including in a non-limiting perspective, Inflammatory Bowel Disease, Metabolic Syndrome, T2D, Asthma, Rheumatoid Arthritis, Psoriasis and Systemic Lupus Erythematosus.

In some embodiments, highly purified (R)-3′-hydroxybutyl (R)-3′-hydroxybutyrate may induce macrophage phenotype switching. The mechanism may induce and influence switching between M1 and M2 macrophages. In some embodiments, BHB may promote the polarization of macrophages towards the M2 phenotype, which may be associated with anti-inflammatory properties. In some embodiments, this effect may be mediated via two pathways; HDAC inhibition and GPT109A signalling. In HDAC Inhibition, BHB-mediated inhibition of histone deacetylases (HDACs) may increase expression of anti-inflammatory cytokines like IL-10 and promote the M2 macrophage phenotype. In GPR109A signalling, activation of the hydroxycarboxylic acid receptor 2 (HCAR2 or GPR109A) by BHB may also contribute to M2 polarization by inhibiting the NF-kB pathway and reducing the production of pro-inflammatory cytokines typical of M1 macrophages. These two pathways may, therefore, have clinical relevance to diseases which may be associated when M1 is the dominant macrophage phenotype. These diseases many include, in a non-limiting sense, Atherosclerosis, Obesity, Rheumatoid Arthritis, Tuberculosis, Cancer, Sepsis, Chronic Obstructive Pulmonary Disease (COPD), Systemic Lupus Erythematosus (SLE), neurodegenerative diseases (e.g., Alzheimer's disease, Parkinson's disease), and Diabetic Wound Healing.

### Highly Purified (R)-3′-hydroxybutyl (R)-3′-hydroxybutyrate and Cognitive Function

In certain examples, the method for enhancing cognitive function in a subject may involve administering an effective amount of highly purified (R)-3′-hydroxybutyl (R)-3′-hydroxybutyrate through oral ingestion. The highly purified (R)-3′-hydroxybutyl (R)-3′-hydroxybutyrate may be prepared as a liquid solution, gel, or encapsulated in a pill to make consumption easier and improve taste. Depending on the subject's needs and the desired cognitive enhancement, administration may occur in a single dose or multiple doses throughout the day.

Alternatively, highly purified (R)-3′-hydroxybutyl (R)-3′-hydroxybutyrate may be delivered via a transdermal patch, providing a controlled release of the active compound over an extended period, which helps maintain steady BHB levels in the blood. This approach may be especially beneficial for subjects who have difficulty with oral ingestion or need a prolonged cognitive boost.

Additionally, highly purified (R)-3′-hydroxybutyl (R)-3′-hydroxybutyrate may be combined with other cognitive enhancers, such as caffeine or nootropic compounds, to potentially enhance the cognitive benefits. The method may be customized for different populations, including healthy individuals seeking improved mental performance, elderly subjects aiming to preserve cognitive function, and patients with cognitive impairments due to psychiatric conditions or neurodegenerative diseases. Cognitive function may be monitored using standardized cognitive tests, EEG measurements, or other neurophysiological assessments to evaluate the treatment's effectiveness.

In some embodiments, the method for improving cognitive performance in subjects with psychiatric conditions may involve administering highly purified (R)-3′-hydroxybutyl (R)-3′-hydroxybutyrate in a liquid form, which may be ingested orally. The highly purified (R)-3′-hydroxybutyl (R)-3′-hydroxybutyrate composition may be formulated to include additional ingredients such as flavor enhancers or stabilizers to improve palatability and shelf-life. The administration may be done daily over a specified period, such as four weeks, to observe significant cognitive improvements. EEG tests are conducted at baseline and at regular intervals during the treatment period to measure changes in brain activity, focusing on specific cognitive domains like attention, memory, and executive function. In another example, the highly purified (R)-3′-hydroxybutyl (R)-3′-hydroxybutyrate comprising composition may be delivered in a capsule or tablet form, providing an alternative for subjects who prefer not to consume liquid supplements. The dosage and frequency of administration may be adjusted based on the severity of the psychiatric condition and the subject's response to the treatment. Additionally, the EEG tests may be complemented with other neuroimaging techniques such as fMRI to provide a more comprehensive assessment of brain function. In a further example, the method may include a combination of highly purified (R)-3′-hydroxybutyl (R)-3′-hydroxybutyrate administration with cognitive behavioral therapy (CBT) sessions, aiming to enhance the overall therapeutic effect. The EEG tests in this case may not only measure brain activity but also track changes in neural connectivity patterns associated with cognitive and emotional regulation. This multi-faceted approach may allow for a more personalized treatment plan, potentially leading to better outcomes for subjects with psychiatric conditions.

In some embodiments, the method for improving cognitive function in elderly subjects may involve administering a highly purified (R)-3′-hydroxybutyl (R)-3′-hydroxybutyrate composition in a liquid form, such as a drink or a solution, which may be easily ingested and absorbed by the body. The composition may include additional ingredients like electrolytes or flavoring agents to enhance palatability and ensure better compliance among elderly subjects. In another example, the highly purified (R)-3′-hydroxybutyl (R)-3′-hydroxybutyrate composition may be administered in a solid form, such as capsules or tablets, which may be preferred for ease of storage and precise dosing. The cognitive tests conducted to measure improvements could vary, including standardized tests for memory recall, executive function, and attention span, tailored to the cognitive abilities and limitations of elderly subjects. Additionally, the method may incorporate the use of wearable devices to monitor physiological parameters such as heart rate variability and sleep patterns, providing a comprehensive assessment of the cognitive and overall health benefits of the highly purified (R)-3′-hydroxybutyl (R)-3′-hydroxybutyrate composition. In a further example, the administration of the highly purified (R)-3′-hydroxybutyl (R)-3′-hydroxybutyrate composition may be combined with a structured cognitive training program, where subjects engage in specific mental exercises designed to enhance cognitive function, thereby potentially amplifying the benefits of the highly purified (R)-3′-hydroxybutyl (R)-3′-hydroxybutyrate. The frequency and duration of administration may also vary, with some protocols involving daily doses while others might use intermittent dosing schedules to optimize cognitive outcomes.

In some embodiments, the method for enhancing cognitive function in subjects with schizophrenia may involve administering a highly purified (R)-3′-hydroxybutyl (R)-3′-hydroxybutyrate composition in a controlled clinical setting, where the dosage and frequency of administration are tailored to the individual needs of the patient based on their medical history and current health status. The highly purified (R)-3′-hydroxybutyl (R)-3′-hydroxybutyrate composition may be delivered in various forms, such as oral capsules, liquid solutions, or even intravenous infusions, depending on the patient's ability to ingest and metabolize the compound effectively. Cognitive tests, such as the Wisconsin Card Sorting Test (WCST) and the Continuous Performance Test (CPT), may be conducted to measure improvements in executive function, attention, and working memory. Additionally, EEG tests may be performed to monitor brain activity, focusing on specific biomarkers associated with cognitive function in schizophrenia, such as gamma oscillations and P300 waveforms. In another example, the method may include a combination therapy approach, where the highly purified (R)-3′-hydroxybutyl (R)-3′-hydroxybutyrate composition may be administered alongside standard antipsychotic medications to evaluate potential synergistic effects on cognitive enhancement. This combination therapy may be monitored through regular blood tests to measure ketone levels and ensure there are no adverse interactions between the ketone ester and the antipsychotic drugs. Furthermore, the method may be adapted to include lifestyle interventions, such as dietary modifications and exercise regimens, to support overall metabolic health and enhance the efficacy of the highly purified (R)-3′-hydroxybutyl (R)-3′-hydroxybutyrate composition. In a third example, the method may incorporate the use of wearable technology to continuously monitor physiological parameters, such as heart rate variability and sleep patterns, providing real-time data to adjust the treatment protocol dynamically. This personalized approach may ensure that the cognitive enhancement strategy is optimized for each individual, considering their physiological responses and lifestyle factors.

In some embodiments, the method for improving cognitive function in subjects with bipolar disorder may involve administering a highly purified (R)-3′-hydroxybutyl (R)-3′-hydroxybutyrate composition in a liquid form, which may be ingested orally. The highly purified (R)-3′-hydroxybutyl (R)-3′-hydroxybutyrate composition may be formulated to include additional ingredients such as electrolytes and flavoring agents to enhance palatability and absorption. The administration may be done daily over a period of several weeks, with cognitive tests conducted at regular intervals to measure improvements in various domains such as memory, attention, and executive function. In another example, the highly purified (R)-3′-hydroxybutyl (R)-3′-hydroxybutyrate composition may be delivered via a slow-release capsule, allowing for a more controlled and sustained release of the active compound. This method may be particularly beneficial for subjects who have difficulty with frequent dosing. Additionally, the cognitive tests may be complemented with EEG tests to monitor brain activity and assess the impact of the ketone ester on neural function. In a further example, the highly purified (R)-3′-hydroxybutyl (R)-3′-hydroxybutyrate composition may be combined with other therapeutic agents, such as mood stabilizers or antipsychotic medications, to provide a synergistic effect on cognitive function. This combination therapy may be tailored to the individual needs of the subject, with dosages adjusted based on periodic assessments of cognitive performance and overall health. The method could also include the use of wearable devices to monitor physiological markers such as heart rate variability and sleep patterns, providing a comprehensive approach to evaluating the benefits of the highly purified (R)-3′-hydroxybutyl (R)-3′-hydroxybutyrate composition on cognitive function in subjects with bipolar disorder.

In some embodiments, the method for enhancing cognitive function in healthy subjects may involve administering a highly purified (R)-3′-hydroxybutyl (R)-3′-hydroxybutyrate composition in a liquid form, such as a drink or a solution, which may be flavored to improve palatability. The composition may include additional ingredients like electrolytes or vitamins to support overall health and enhance the cognitive benefits. In another example, the highly purified (R)-3′-hydroxybutyl (R)-3′-hydroxybutyrate composition is provided in a solid form, such as capsules or tablets, which may be taken orally with water. This form may be preferred for individuals who require precise dosing or have dietary restrictions. Additionally, the method may be adapted to different administration schedules, such as a single daily dose or multiple smaller doses throughout the day, depending on the subject's needs and lifestyle. The cognitive function improvements may be measured using a variety of validated cognitive tests, including those assessing executive function, memory, attention, and reaction times. Furthermore, the method may be tailored to different age groups, with specific formulations and dosages designed for younger adults, middle-aged individuals, and the elderly, ensuring that the cognitive benefits are optimized for each demographic. The highly purified (R)-3′-hydroxybutyl (R)-3′-hydroxybutyrate composition may also be combined with other nootropic agents or cognitive enhancers to create a synergistic effect, potentially leading to greater improvements in cognitive performance. The method may include monitoring biomarkers such as blood ketone levels, serum PH, metabolic markers, and neuroprotective markers to assess the efficacy and safety of the highly purified (R)-3′-hydroxybutyl (R)-3′-hydroxybutyrate composition over time.

### Highly Purified (R)-3′-hydroxybutyl (R)-3′-hydroxybutyrate and Neurodegenerative Diseases

In some embodiments, the method for treating neurodegenerative diseases may involve administering a highly purified (R)-3′-hydroxybutyl (R)-3′-hydroxybutyrate composition formulated to enhance neuroprotective effects. This composition may include highly purified (R)-3′-hydroxybutyl (R)-3′-hydroxybutyrate, which may be metabolized to generate BHB, which has been shown to improve mitochondrial function and reduce oxidative stress. The administration may be done orally, through a liquid or capsule form, ensuring ease of use for patients. In another example, the highly purified (R)-3′-hydroxybutyl (R)-3′-hydroxybutyrate composition may be combined with other neuroprotective agents, such as antioxidants or anti-inflammatory compounds, to provide a synergistic effect. This combination may be tailored based on the neurodegenerative condition being treated, such as Alzheimer's disease, Parkinson's disease, or amyotrophic lateral sclerosis (ALS). Additionally, the treatment regimen may involve varying dosages and frequencies of administration, depending on the severity of the disease and the patient's response to the treatment. Monitoring neuroprotective markers in the blood, such as brain-derived neurotrophic factor (BDNF) and inflammatory cytokines, may be a part of the treatment protocol to assess efficacy and make helpful adjustments. Furthermore, the method may be adapted to include non-invasive brain imaging techniques, like functional MRI or PET scans, to observe changes in brain activity and structure over time, providing a comprehensive approach to managing neurodegenerative diseases.

In some embodiments, the method for treating neurodegenerative diseases may involve administering a highly purified (R)-3′-hydroxybutyl (R)-3′-hydroxybutyrate composition formulated to enhance neuroprotective effects. This composition may include a purified R-form enantiomer of a ketone ester, which has been shown to improve mitochondrial function and reduce oxidative stress. The administration may be done orally, through a liquid or capsule form, ensuring ease of use for patients. In another example, the highly purified (R)-3′-hydroxybutyl (R)-3′-hydroxybutyrate composition may be combined with other neuroprotective agents, such as antioxidants or anti-inflammatory compounds, to provide a synergistic effect. This combination may be tailored based on the neurodegenerative condition being treated, such as Alzheimer's disease, Parkinson's disease, or amyotrophic lateral sclerosis (ALS). Additionally, the treatment regimen may involve varying dosages and frequencies of administration, depending on the severity of the disease and the patient's response to the treatment. Monitoring neuroprotective markers in the blood, such as brain-derived neurotrophic factor (BDNF) and inflammatory cytokines, may be a part of the treatment protocol to assess efficacy and make helpful adjustments. Furthermore, the method may be adapted to include non-invasive brain imaging techniques, like functional MRI or PET scans, to observe changes in brain activity and structure over time, providing a comprehensive approach to managing neurodegenerative diseases.

In some embodiments, the use of highly purified (R)-3′-hydroxybutyl (R)-3′-hydroxybutyrate in a treatment protocol may involve numerous mechanisms or effects. In a non-limiting example, BHB may serve as an alternative energy source. In another example, BHB may act by reducing oxidative stress in the user. In some non-limiting examples, BHB decreases the production of reactive oxygen species (ROS) by increasing the Q/QH2 ratio in the electron transport chain, which reduces reverse electron transport and consequently the generation of superoxide radicals. Additionally, BHB may enhance the NADP+/NADPH ratio, supporting the synthesis of antioxidants like glutathione, thioredoxins, and vitamins C and E. For these effects there may be numerous disease states of clinical relevance that may be improved including, in a non-limiting perspective, Alzheimer's disease, Parkinson's disease, Huntington's disease, Amyotrophic Lateral Sclerosis (ALS)/Motor Neuron Disease, and Multiple sclerosis. In some other examples, other rarer disease states that may be improved include Friedreich's Ataxia, Creutzfeldt-Jakob Disease, Spinocerebellar Ataxia, Lewy Body Dementia, and Progressive Supranuclear Palsy. Apart from the neurodegenerative diseases, the reduction of oxidative stress in neurons may also be relevant for TBI and metabolic psychiatry (autism, bipolar disease, or schizophrenia). As part of a treatment methodology, there may be numerous biomarkers that may be used for assessment. These biomarkers to assess the antioxidative effects may include Isoprostanes, which may be common plasma markers of lipid oxidation, or 7,8-dihydro-8-oxo-2′-deoxyguanosine, which may be a marker of DNA oxidative damage (part of the SOS panel). The SOS panel also includes “reactive oxygen metabolites” marker. Other markers may include Thiobarbituric Acid Reducing Substances (TBARS) or 8-Iso-Prostaglandin-F2α (F2-IsoPs), and Protein carbonyls as non-limiting cases.

In some embodiments, the use of highly purified (R)-3′-hydroxybutyl (R)-3′-hydroxybutyrate may involve a mechanism wherein generated BHB may induce anti-inflammatory effects. In some embodiments, BHB may exert anti-inflammatory effects by binding to hydroxycarboxylic acid receptor 2 (HCAR2), which inhibits the nuclear factor-kappa B (NF-κB) pathway. This inhibition may reduce the expression of pro-inflammatory cytokines such as TNF-α and IL-1β, and suppresses inflammasome activation, particularly the NLRP3 inflammasome, thereby reducing neuroinflammation. There may be numerous disease states where such effects of the treatment with highly purified (R)-3′-hydroxybutyl (R)-3′-hydroxybutyrate may have clinical relevance including in a non-limiting perspective: Alzheimer's, Parkinson's, ALS, Huntington's disease, multiple sclerosis, frontotemporal dementia or progressive supranuclear palsy. There may also be links between neuroinflammation and TBI and metabolic psychiatry (autism, bipolar disease or schizophrenia). As part of a treatment protocol there may be measurements of various biomarkers including serum levels of IL-1beta, IL-6, IL-10, TNF-alpha and C-reactive protein (CRP) for example.

In some embodiments, the use of highly purified (R)-3′-hydroxybutyl (R)-3′-hydroxybutyrate may involve a mechanism wherein BHB may mediate enhancement of mitochondrial function. In some embodiments, BHB may improve mitochondrial function by increasing the activity of enzymes like Peroxisome Proliferator-Activated Receptor Gamma Coactivator 1-alpha (PGC-1α), which facilitates mitochondrial biogenesis. BHB may also stimulate the deacetylation of transcription factors such as Sirtuin 1 (SIRT1), enhancing the expression of antioxidant genes and improving the health of the mitochondrial pool.

In some embodiments, the use of highly purified (R)-3′-hydroxybutyl (R)-3′-hydroxybutyrate may involve a mechanism wherein BHB may induce autophagy. BHB may stimulate autophagic flux, which may be helpful for degrading and recycling damaged cellular components, including defective mitochondria. This process may be regulated through pathways involving SIRT1, AMP-activated protein kinase (AMPK), Unc-51-like kinase 1(ULK1), and mTORC1. By enhancing autophagy, BHB may help maintain cellular homeostasis and prevents the accumulation of toxic protein aggregates. These improvements may be useful in treatment of disease states such as Alzheimer's amongst others. There may be other methods of treatment, which may include assessment of biomarkers to regulate the treatment. These biomarkers may include, in a non-limiting perspective, serum level of ATG5 protein or Parkin proteins.

In some embodiments, the use of highly purified (R)-3′-hydroxybutyl (R)-3′-hydroxybutyrate may involve a mechanism wherein BHB may regulate apoptosis. BHB may modulate apoptotic pathways by inhibiting pro-apoptotic factors like p53 and promoting the expression of anti-apoptotic proteins. This regulation may reduce neuronal apoptosis and may support cell survival under stress conditions. These improvements may be useful in treatment of disease states such as Alzheimer's, Huntington's, Parkinson's, ALS, TBI and stroke amongst others. There may be additional methods of treatment, which may include assessment of biomarkers to regulate the treatment, these biomarkers may include in a non-limiting perspective caspase-3 in cerebrospinal fluid is a marker of neuronal apoptosis after strokes or TBI. For TBI serum levels of S100B and GFAP may be used as biomarkers, In some embodiments.

### Highly Purified (R)-3′-hydroxybutyl (R)-3′-hydroxybutyrate and Metabolic Disorders

Given its function as an alternative cerebral energy substrate to glucose during restricted dietary access to carbohydrates, R-BHB may be inherently associated with states of low-calorie availability. This may make highly purified (R)-3′-hydroxybutyl (R)-3′-hydroxybutyrate a good candidate as a molecule not only to provide an alternative source of energy but also to elicit various cellular processes, which facilitate the adaptation to low-calorie states. In some embodiments, calorie restriction may alleviate a wide range of non-communicable disease states, it may be expected that compounds which may mimic the adaptations to stresses of low calories may also prevent and treat diseases, BHB generated from highly purified (R)-3′-hydroxybutyl (R)-3′-hydroxybutyrate may therefore be considered to be a calorie restriction mimetic.

The set of adaptations required for the reprogramming of the metabolic profile of cells and organs may be extensive. Similarly, the signaling effects of BHB may also be relatively broad, which may make highly purified (R)-3′-hydroxybutyl (R)-3′-hydroxybutyrate well-positioned to act as a multi-target therapeutic.

In some embodiments, BHB may act to improve metabolic disorders through a mechanism of increasing AMPK activity. In some embodiments, AMPK may act as a sensor of cellular energy. Once it is activated, AMPK may restore energy balance by promoting energy-generating processes and inhibiting energy-consuming processes. For example, this inhibition may come through increased mitochondrial biogenesis, activated autophagy, inhibition of the mechanistic Target of Rapamycin (mTOR) complex or promotion of uptake of glucose from bloodstream independently of insulin.

For example, a specific type of post-translational modification of histone proteins, which may be referred to as Kbhb, may involve the addition of a BHB group to lysine residues on histone proteins, which DNA wraps around. By changing the chromatin structure through the Kbhb mechanism, the accessibility of DNA sequences may changes. Accordingly, Kbhb may result in changes to regulation of gene expression. In some studies, the genes affected by Kbhb have been characterized. Downregulated genes were mainly involved in glycolysis and ribosome assembly pathways, while upregulated genes were involved in mitochondrial metabolism.

In some other examples, Kbhb may also occur on lysine residues of nonhistone proteins. In some embodiments, Kbhb associated proteins may be involved in spliceosome, ribosome, and RNA transport functions. There also may also be a role for Kbhb associated proteins in DNA repair-related pathways, such as nucleotide excision repair, mismatch repair, and base excision repair. In some embodiments, these Kbhb associated proteins may be highly enriched, with 43, 57, and 39% of proteins in these pathways being beta-hydroxybutyrylated.

In a different set of examples, highly purified (R)-3′-hydroxybutyl (R)-3′-hydroxybutyrate may act to improve metabolic disorders through a mechanism involving generated BHB and histone deacetylase (HDAC) inhibition. HDAC refers to a process which removes acetyl groups from histone proteins. In a similar manner to the process of Kbhb, the inhibition of histone deacetylation may change the structure of the chromatin and, accordingly, may influence gene expression. In some embodiments, numerous signaling effects of BHB are mediated by HDAC inhibition. In a non-limiting example, the activation of Forkhead Box O3 (FOXO3) transcription factor by BHB may be facilitated by HDAC inhibition.

In a different set of examples, highly purified (R)-3′-hydroxybutyl (R)-3′-hydroxybutyrate may act to BHB to improve metabolic disorders through a mechanism involving regulation of inflammation such as through NOD-like receptor family pyrin domain containing 3 (NLRP3) protein inhibition by BHB. In some embodiments, NLRP3 may be an inflammasome that facilitates the activation of pro-inflammatory cytokine. In similar examples, Nuclear Factor kappa B (NF-kappaB) proteins may be inhibited by BHB. NF-kappaB may control the production of pro-inflammatory cytokines.

In a still further different set of examples, highly purified (R)-3′-hydroxybutyl (R)-3′-hydroxybutyrate may act through generated BHB to improve metabolic disorders through a mechanism involving increased mitochondrial biogenesis. In some embodiments, R-BHB may upregulate expression of PGC-1alpha. PGC-1alpha may be a master regulator of mitochondrial biogenesis.

In additional examples, highly purified (R)-3′-hydroxybutyl (R)-3′-hydroxybutyrate may act to improve metabolic disorders through generated BHB through a mechanism involving the induction of autophagy. In some embodiments, BHB treatment may induce autophagic flux, as related to the upregulation of autophagy-related proteins like BECN1 and the downregulation of p62, an autophagy adaptor protein.

In some embodiments, highly purified (R)-3′-hydroxybutyl (R)-3′-hydroxybutyrate may act through generated BHB to improve metabolic disorders through a mechanism involving enhancement of Brain-Derived Neurotrophic Factor (BDNF) expression. In some embodiments, R-BHB may increase BDNF expression. The primary function of BDNF may be to support the survival, growth, and maintenance of neurons in the brain and peripheral nervous system.

In some embodiments, highly purified (R)-3′-hydroxybutyl (R)-3′-hydroxybutyrate may act through generated BHB to improve metabolic disorders through a mechanism involving activation of hydroxycarboxylic acid receptor 2 (HCAR2). This receptor may also be known as PUMA-G and GPR109A, and its activation may inhibit lipolysis in adipocytes, lower plasma FFA and glucose levels in individuals with type 2 diabetes and contribute to neuroprotection in the brain.

In some embodiments, highly purified (R)-3′-hydroxybutyl (R)-3′-hydroxybutyrate may act through generated BHB to improve metabolic disorders through a mechanism involving promotion of Vascular Endothelial Growth Factor (VEGF) production. VEGF may be a master regulator of vascular growth, and BHB was shown to promote VEGF production. In some of these examples, such an effect may alleviate diabetic aortic endothelial injury as a non-limiting example.

In some embodiments, highly purified (R)-3′-hydroxybutyl (R)-3′-hydroxybutyrate may act through BHB to improve metabolic disorders through a mechanism involving prevention of DNA damage by the alleviation of oxidative stress. Oxidation of BHB may reduce oxidative stress by replenishing coenzyme Nicotinamide Adenine Dinucleotide Phosphate (NADPH) levels and increasing the Ubiquinone (Q also called CoQ10) to Ubiquinol (QH2—a reduced form of Ubiquinone) ratio. Moreover, BHB may also stimulate the activity of FOXO3a (an isoform of FOX03) via HDAC inhibition and Nrf2 activity, which results in the expression of endogenous antioxidants like catalase, Glutamate-Cysteine Ligase (GCL) and Manganese Superoxide Dismutase (mnSOD).

In some embodiments, highly purified (R)-3′-hydroxybutyl (R)-3′-hydroxybutyrate may act through generated BHB to improve metabolic disorders through a mechanism involving affecting biochemical pathways, which regulate cellular senescence. BHB may react to form beta-hydroxybutyrylate lysine residues on various proteins, including the p53 protein. The beta-hydroxybutyrylation may attenuate Tumor Protein 53 (p53) activity and downregulates levels of Tumor Protein 21 (p21). Another study showed that BHB may mitigate senescence in the kidney by decreasing the expression of Tumor Protein 16 (p16 ) and p21. In vascular cells, BHB prevented senescence by increasing Lamin B1 via stabilization of Octamer-binding transcription factor 4 (Oct 4 mRNA).

In some embodiments, the method for treating metabolic disorders in a subject may involve administering a combination of highly purified (R)-3′-hydroxybutyl (R)-3′-hydroxybutyrate and an anti-diabetic drug or R-BHB and an anti-diabetic drug such as metformin, to the subject. In other examples, the combination may be with one or more of the following materials including diuretics and glucagon like protein-1 receptor agonists including but not limited to semaglutide, liraglutide, exenatide, dulaglutide, lixisenatide, albiglutide and efpeglenatide. In some other examples, highly purified (R)-3′-hydroxybutyl (R)-3′-hydroxybutyrate may be combined with dual glucagon like protein-1 receptor agonist and glucose-dependent insulinotropic polypeptide receptor agonist molecules such as tirzepatide.

The highly purified (R)-3′-hydroxybutyl (R)-3′-hydroxybutyrate will generate a purified R-form enantiomer of BHB, which enhances the metabolic benefits by providing a more efficient energy source and reducing oxidative stress. The anti-diabetic drug may be administered in various forms, including oral tablets, capsules, or injectable solutions, depending on the subject's specific needs and medical conditions. In another example, highly purified (R)-3′-hydroxybutyl (R)-3′-hydroxybutyrate may be combined with a different class of anti-diabetic drugs, such as SGLT2 inhibitors including but not limited to Canagliflozin, Dapagliflozin, Empagliflozin, and Ertugliflozin, to further enhance glucose regulation and improve insulin sensitivity.

The administration may be tailored to the subject's daily routine, with highly purified (R)-3′-hydroxybutyl (R)-3′-hydroxybutyrate being taken in the morning to provide sustained energy throughout the day, and the anti-diabetic drug being taken in the evening to optimize blood glucose levels overnight. Additionally, the method may include monitoring the subject's metabolic markers, such as blood glucose levels, HbA1c, and lipid profiles, to adjust the dosage and combination of highly purified (R)-3′-hydroxybutyl (R)-3′-hydroxybutyrate and anti-diabetic drug for maximum therapeutic efficacy. This approach allows for a personalized treatment plan that addresses the metabolic needs of each subject, potentially improving their overall metabolic health and quality of life.

In some embodiments, the method for treating metabolic disorders in a subject may involve administering a combination of highly purified (R)-3′-hydroxybutyl (R)-3′-hydroxybutyrate and an anti-diabetic drug, such as metformin, to enhance the therapeutic effects on glucose regulation and insulin sensitivity. The ketone ester may be administered in various forms, including oral capsules, liquid solutions, or transdermal patches, to accommodate different patient preferences and medical needs. In another example, highly purified (R)-3′-hydroxybutyl (R)-3′-hydroxybutyrate may be combined with other anti-diabetic drugs like sulfonylureas or DPP-4 inhibitors, providing a tailored approach to managing different types of diabetes and metabolic conditions. The administration schedule may vary, with some protocols involving daily doses while others may use intermittent dosing to optimize metabolic benefits and minimize potential side effects. Additionally, the method may include monitoring metabolic markers such as blood glucose levels, HbA1c, and lipid profiles to assess the treatment's efficacy and make helpful adjustments. In a further example, the highly purified (R)-3′-hydroxybutyl (R)-3′-hydroxybutyrate and anti-diabetic drug combination may be integrated into a comprehensive treatment plan that includes dietary modifications and physical exercise, thereby promoting overall metabolic health and improving patient outcomes. This approach allows flexibility in treatment regimens, catering to individual patient needs and enhancing the adaptability of the method within the defined legal and functional scope.

In some embodiments, the method for improving metabolic health may involve administering a highly purified (R)-3′-hydroxybutyl (R)-3′-hydroxybutyrate composition to a subject and monitoring metabolic markers such as blood glucose levels, insulin sensitivity, and lipid profiles. The highly purified (R)-3′-hydroxybutyl (R)-3′-hydroxybutyrate composition may be formulated in various delivery forms, including oral capsules, liquid solutions, or transdermal patches, to accommodate different patient preferences and medical needs. In another example, the highly purified (R)-3′-hydroxybutyl (R)-3′-hydroxybutyrate composition is combined with dietary interventions, such as a ketogenic diet, to enhance the metabolic benefits. This combination may be tailored to individual metabolic profiles, ensuring personalized treatment plans. Additionally, the method may be adapted to include regular physical activity as part of the regimen, with the highly purified (R)-3′-hydroxybutyl (R)-3′-hydroxybutyrate composition administered pre-or post-exercise to optimize metabolic responses. In a further example, the method incorporates the use of wearable technology to continuously monitor metabolic markers such as glucose levels and ketone concentrations in real-time, providing immediate feedback and allowing for dynamic adjustments to the treatment protocol. This example may also include periodic blood tests to measure more comprehensive metabolic markers, such as HbA1c and inflammatory cytokines, to assess long-term metabolic health improvements. Each of these examples demonstrates the adaptability of the method within the legal and functional scope defined by the patent claims, ensuring a broad application for improving metabolic health in various subject populations.

In some embodiments, the use of highly purified (R)-3′-hydroxybutyl (R)-3′-hydroxybutyrate may involve a mechanism wherein generated BHB may suppress appetite. BHB may reduce levels of the hunger hormone ghrelin. Reduction in appetite might decrease the risk of obesity and all the diseases associated with obesity-type 2 Diabetes, Cardiovascular Disease, Hypertension, Osteoarthritis, Sleep Apnoea, Non-Alcoholic Fatty Liver Disease. There may be methods of treatment which may include assessment of biomarkers to regulate the treatment, these biomarkers may include in a non-limiting perspective measurement of HbA1C, Fructosamine, serum glucose, C-Reactive Protein (CRP), high-sensitivity CRP (hs-CRP), serum IL-6, TNF-α, Fibrinogen, and D-dimer levels.

In some embodiments, the use of highly purified (R)-3′-hydroxybutyl (R)-3′-hydroxybutyrate may involve a mechanism wherein generated BHB may suppress levels of lipids in serum. In some embodiments, BHB may inhibit lipolysis, a process by which fats are broken down into free fatty acids (FFA) and glycerol. This inhibition may occur via the activation of the HCAR2 receptor (also known as GPR109A), which is a G-protein coupled receptor that reduces the activity of the adenylate cyclase/cAMP/PKA signalling pathway. This reduction may lead to decreased lipolytic activity in adipocytes, resulting in lower levels of circulating free fatty acids, which are precursors for triglyceride and cholesterol synthesis. Accordingly, highly purified (R)-3′-hydroxybutyl (R)-3′-hydroxybutyrate supplementation may lower LDL levels. Furthermore, highly purified (R)-3′-hydroxybutyl (R)-3′-hydroxybutyrate supplementation may increase HDL cholesterol in some embodiments. There may be methods of treatment which may include assessment of biomarkers to regulate the treatment, these biomarkers may include in a non-limiting perspective measurement of triglycerides, LDL, v-LDL, and HDL cholesterol, Apolipoprotein B (ApoB), and Oxidized LDL levels.

In some embodiments, the use of highly purified (R)-3′-hydroxybutyl (R)-3′-hydroxybutyrate may involve a mechanism wherein generated BHB may prevent liver fat accumulation and Non-Alcoholic Fatty Liver Disease (NAFLD) progression. Excessive accumulation of triacylglycerol (TAG) in NAFLD may be caused by an imbalance between increased hepatic free fatty acids (FFA) uptake and decreased FFA removal via beta-oxidation, lipophagy and exportation via very-low-density lipoprotein. Insulin resistance (IR) may be a helpful factor in NAFLD because it may increase peripheral fat lipolysis, making FFA available for hepatic uptake. This may enhance hepatic lipogenesis. A master regulator of FFA oxidation in the liver may be the Peroxisome Proliferator-Activated Receptor Alpha (PPARα) nuclear receptor protein. Upon activation, PPARα may stimulate the expression of genes involved in FFA uptake, beta-oxidation and triglyceride turnover. PPARα levels from liver biopsies of NAFLD patients negatively correlate with the severity of steatosis and fibrosis. Conversely, an increase in PPARα was observed after improvement of NAFLD caused by lifestyle modification or bariatric surgery. Accordingly, PPARα agonists may have a use as anti-NAFLD drugs. The activity of PPARα is augmented by its co-activator Peroxisome Proliferator-Activated Receptor Gamma Coactivator 1-alpha (PGC-1α), which may increase expression of mitochondrial fatty acid oxidation enzymes. PGC-1α overexpression may increase hepatic mitochondrial content and decrease hepatic TAG storage and TAG plasma levels. Increased PGC-1α expression may also compensate for the depletion of mitochondrial DNA observed in NAFLD. Moreover, excess lipid accumulation in the liver may induce endoplasmic reticulum (ER) stress. In turn, ER stress may further dysregulate lipid metabolism by stimulating lipogenesis due to greater SREBP activity.

In some embodiments, elevated levels of extracellular succinate may be a pro-inflammatory driver of NAFLD. Succinate Receptor 1(SUCNR1 ) may be expressed on the surface of circulating immune cells and also on liver-residing macrophages and on hepatic stellate cells (HSC). The activation of SUCNR1 on HSC by succinate may be an important stimulator of fibrosis. Moreover, activation of SUCNR1 on immune cells may lead to the expression of pro-inflammatory cytokine IL-1β, which may promote liver steatosis and fibrosis through the IL-1 receptor. In some embodiments, liver biopsies may be used to demonstrate that IL-1β is elevated in NAFLD patients and IL-1β mRNA levels may be significantly correlated with Collagen Type I Alpha 1 Chain (COL1A1), a key fibrogenic gene. In some embodiments, NLRP3 inflammasome may be important in the progression of NAFLD into non-alcoholic steatohepatitis (NASH).

In some embodiments, the liver may not use (R)-3′-hydroxybutyl (R)-3′-hydroxybutyrate as an energy substrate because of the lack of β-ketoacyl-CoA transferase in hepatocytes. However, hepatocytes express MCT1, which may transport BHB to the liver and BHB signaling effects may alleviate NAFLD even without BHB oxidation. BHB was reported to increase the expression of PGC-1α, which enhances the activity of PPARα and improves mitochondrial function. The mitochondrial damage may also be alleviated by BHB's ability to activate FOXO3, a transcription factor regulating major endogenous antioxidants like manganese superoxide dismutase (mnSOD) catalase.

Moreover, a ketone ester diet (KED) in particular including ingesting of highly purified (R)-3′-hydroxybutyl (R)-3′-hydroxybutyrate may stimulate mitochondrial biogenesis, increasing PGC-1α expression and increasing the expression of Uncoupling Protein 1 (UCP1), thereby, increasing its metabolic activity. Increasing the metabolic activity of Brown Adipose Tissue (BAT) enhances its ability to uptake succinate and ameliorates NAFLD pathology. Decreasing activity of SUCNR1 by BAT-mediated succinate uptake decreased the expression of inflammatory cytokines like IL-1β, IL-6 and TNFα. BHB also may further decrease NALFD-related inflammation and progression via inhibition of NLRP3 inflammasome, which may decrease levels of fibrogenic IL-1β.

In some embodiments, reduction of ER stress with highly purified (R)-3′-hydroxybutyl (R)-3′-hydroxybutyrate ingestion may be mediated by increasing the activity of the AMPK-FOXO3 pathway. Activation of AMPK-FOXO3 may result in increased levels of antioxidants mnSOD and catalase. In some embodiments, highly purified (R)-3′-hydroxybutyl (R)-3′-hydroxybutyrate ingestion may improve insulin signaling and show improved scores of homeostatic model assessment for IR and lower HbA1c. IR may also be improved via weight loss and lifestyle changes. In some embodiments, BHB may suppress appetite in humans by lowering ghrelin levels, which might positively support weight loss efforts.

Amelioration of NAFLD may reduce risk for all-cause mortality and decrease the risk of liver diseases including progression to NASH, Liver fibrosis and cirrhosis. There may be methods of treatment with highly purified (R)-3′-hydroxybutyl (R)-3′-hydroxybutyrate, which may include assessment of biomarkers to regulate the treatment, these biomarkers may include in a non-limiting perspective, MRI-assessed liver steatosis, measurement of serum levels of tissue inhibitor of metalloproteinases 1, serum levels of cytokeratin 18 fragment levels and fibroblast growth factor 21 levels. There may be other supporting markers such as the standard lipid panel for LDL, HDL, and triglycerides, and measurement of plasma alkaline phosphatase, alanine transaminase, aspartate aminotransferase, and albumin.

In some embodiments, highly purified (R)-3′-hydroxybutyl (R)-3′-hydroxybutyrate ingestion may induce fat browning. Brown adipocytes may oxidize fatty acids due to high mitochondrial density and uncoupled cellular respiration from ATP synthesis, which produces heat. Activation of brown fat induces thermogenesis, increases calorie output, and may reduce obesity and other metabolic diseases associated with overconsumption. Fat browning may be induced by highly purified (R)-3′-hydroxybutyl (R)-3′-hydroxybutyrate ingestion to generate BHB by increasing expression of UCP1 protein, which is responsible for the uncoupling of cellular respiration and ATP production. Study in rats showed that BHB is capable of inducing. Fat browning may have clinical relevance to numerous disease state including in a non-limiting perspective obesity-type 2 Diabetes, Cardiovascular Disease, Hypertension, Osteoarthritis, Sleep Apnea, Non-Alcoholic Fatty Liver Disease. There may be methods of treatment with highly purified (R)-3′-hydroxybutyl (R)-3′-hydroxybutyrate, which may include assessment of biomarkers to regulate the treatment, these biomarkers may include in a non-limiting perspective, exosomal microRNA miR-92a concentration in serum reflect human brown fat activity. In some embodiments, biopsy of adipose tissues may also be possible to assess UCP1 expression. As well, regulation of treatment may include measurements of serum levels of UCP1 itself as well as measurements of serum levels of Fibroblast Growth Factor 21 (FGF21).

### Highly Purified (R)-3′-hydroxybutyl (R)-3′-hydroxybutyrate and Sarcopenia

In some embodiments, highly purified (R)-3′-hydroxybutyl (R)-3′-hydroxybutyrate ingestion may ameliorate the symptoms of Sarcopenia, which relates to age-related loss of muscle mass, strength. Ingestion of highly purified (R)-3′-hydroxybutyl (R)-3′-hydroxybutyrate may act by generating BHB, which may act through effecting the mTOR pathway. BHB may activate the mTORC1 (mechanistic target of rapamycin complex 1) signaling pathway, which may be helpful for initiating muscle protein synthesis. mTORC1 may promote the phosphorylation of downstream targets such as p70S 6 kinase (p70S6K) and 4E-BP1. The phosphorylation of p70S6K may enhance the translation of mRNAs required for muscle protein synthesis. This cascade may ultimately lead to increased muscle protein synthesis, counteracting muscle loss in sarcopenia. In some embodiments, the relationship between mTOR activation and sarcopenia may be complex and may lead to different strategies in different cases. Some findings may indicate that while mTORC1 is activated in aging muscle, the effectiveness of this activation in promoting muscle hypertrophy is diminished, leading to the conclusion that low mTOR activation may indeed play a role in sarcopenia. There may be methods of treatment with highly purified (R)-3′-hydroxybutyl (R)-3′-hydroxybutyrate, which may include assessment of biomarkers to regulate the treatment, these biomarkers may include in a non-limiting perspective, biopsy related measurement of levels of phosphorylation of p70S6K, p4EBP1 or pS6.

In some embodiments, highly purified (R)-3′-hydroxybutyl (R)-3′-hydroxybutyrate ingestion may ameliorate the symptoms of Sarcopenia and may act through effecting reduction of inflammation via NF-κB inhibition. In some embodiments, generated BHB may inhibit the NF-κB (nuclear factor kappa-light-chain-enhancer of activated B cells) signaling pathway, which may be an important mediator of inflammation. NF-κB may typically be activated in response to inflammatory cytokines such as TNF-α and IL-1β. Upon activation, NF-κB may translocate to the nucleus and may promote the transcription of pro-inflammatory genes, including those that encode cytokines, chemokines, and adhesion molecules. By inhibiting NF-κB activation, BHB may reduce the expression of these pro-inflammatory mediators, thus lowering systemic inflammation and preventing muscle wasting that may be exacerbated by chronic inflammation. In some embodiments, the relationship between mTOR activation and sarcopenia may be complex and may lead to different strategies in different cases. There may be methods of treatment with highly purified (R)-3′-hydroxybutyl (R)-3′-hydroxybutyrate, which may include assessment of biomarkers to regulate the treatment, these biomarkers may include in a non-limiting perspective, measurement of serum levels of IL-1beta, IL-6, IL-10, TNF-alpha and CRP.

In some embodiments, highly purified (R)-3′-hydroxybutyl (R)-3′-hydroxybutyrate ingestion may ameliorate the symptoms of Sarcopenia and may act through mitigation of oxidative stress by generated BHB. BHB may exert antioxidant effects by enhancing the activity of antioxidant enzymes and reducing the production of reactive oxygen species (ROS). It may stimulate the Nrf2 (nuclear factor erythroid 2-related factor 2) pathway, which may lead to the upregulation of various antioxidant proteins such as superoxide dismutase (SOD) and catalase. Nrf2 activation may also reduce oxidative damage to muscle cells by neutralizing ROS, which may be implicated in the pathogenesis of muscle atrophy and sarcopenia. Additionally, BHB may directly scavenge ROS, further protecting muscle tissues from oxidative damage. There may be methods of treatment with highly purified (R)-3′-hydroxybutyl (R)-3′-hydroxybutyrate, which may include assessment of biomarkers to regulate the treatment, these biomarkers may include in a non-limiting perspective, measurement of Isoprostanes as a are common plasma markers of lipid oxidation, and 7,8-dihydro-8-oxo-2′-deoxyguanosine as a marker of DNA oxidative damage (part of the SOS panel). The SOS panel may also include “reactive oxygen metabolites” marker. In some embodiments, the biomarkers may include in a non-limiting perspective, thiobarbituric acid reducing substances (TBARS) or 8-Iso-Prostaglandin-F2α (F 2-IsoPs).

### Highly Purified (R)-3′-hydroxybutyl (R)-3′-hydroxybutyrate and Mitochondrial Function

In some embodiments, the method for enhancing mitochondrial function in a subject may involve administering a composition containing highly purified (R)-3′-hydroxybutyl (R)-3′-hydroxybutyrate through oral ingestion. The composition may be formulated as a liquid, capsule, or powder to suit different preferences and improve compliance. The dosage may be adjusted based on the subject's weight, age, and health status to ensure optimal efficacy. In another example, the administration may be via intravenous injection for subjects requiring rapid intervention or those with gastrointestinal absorption issues. The highly purified (R)-3′-hydroxybutyl (R)-3′-hydroxybutyrate may be combined with other mitochondrial enhancers such as Coenzyme Q10 or PQQ (Pyrroloquinoline quinone) to synergistically boost mitochondrial biogenesis. Additionally, the method includes measuring mitochondrial biogenesis markers such as PGC-1α, NRF1, and Transcription Factor A, Mitochondrial (TFAM) in blood or muscle tissue samples to monitor the effectiveness of the treatment. In a further example, the composition may be tailored to include specific nutrients like magnesium and B vitamins that support mitochondrial function, providing a comprehensive approach to enhancing cellular energy production. The method may also incorporate periodic assessments using advanced imaging techniques like MitoTracker staining or electron microscopy to visualize changes in mitochondrial density and morphology, ensuring a thorough evaluation of the treatment's impact.

### Highly Purified (R)-3′-hydroxybutyl (R)-3′-hydroxybutyrate and Muscle Power Output

In some embodiments, highly purified (R)-3′-hydroxybutyl (R)-3′-hydroxybutyrate ingestion may be useful in maintaining or enhancing muscle power output through generation of BHB. Specifically, ingestion may be of highly purified (R)-3′-hydroxybutyl (R)-3′-hydroxybutyrate, which may be effective in increasing circulating BHB concentrations in the blood plasma, thereby supporting or improving muscle power output.

BHB may be generated when fatty acid levels rise in the body and are metabolized for energy. They have been found to reduce circulating free fatty acids in the plasma, leading to various health benefits, such as improved cognitive performance, and treatment for cardiovascular conditions, diabetes, mitochondrial dysfunctions, and muscle fatigue.

In examples using highly purified (R)-3′-hydroxybutyl (R)-3′-hydroxybutyrate, beneficial factors may include improved taste aspects and high uptake efficiency. In some embodiments it may be a preferred candidate for achieving significant blood ketone concentrations upon oral ingestion.

In some embodiments, highly purified (R)-3′-hydroxybutyl (R)-3′-hydroxybutyrate may include a variety of forms, such as liquids, powders, tablets, bars, or granules. These compositions may be ingested either in these forms or mixed with liquids like water, juice, or milk, to achieve the desired ketone levels and muscle power benefits.

In some embodiments, compositions may also include additional ingredients such as flavors, proteins, carbohydrates, vitamins, and minerals, to enhance desirability for users.

### Highly Purified (R)-3′-hydroxybutyl (R)-3′-hydroxybutyrate and Cardiovascular Conditions

In some embodiments, the method for treating cardiovascular conditions may involve administering a highly purified (R)-3′-hydroxybutyl (R)-3′-hydroxybutyrate composition in a liquid form, such as a drink or an intravenous solution, to ensure rapid absorption and bioavailability. The composition may include additional ingredients like electrolytes to support cardiovascular health and hydration. In another example, the highly purified (R)-3′-hydroxybutyl (R)-3′-hydroxybutyrate composition is administered in a solid form, such as capsules or tablets, which may be coated to enhance stability and control the release rate of the active ingredient. This form may be particularly useful for long-term treatment regimens. Additionally, the method may include monitoring cardiovascular indicators such as blood pressure, heart rate, and lipid levels through wearable devices that continuously transmit data to healthcare providers. In another example, the highly purified (R)-3′-hydroxybutyl (R)-3′-hydroxybutyrate composition may be administered in combination with cardiovascular medications, such as statins or beta-blockers, to enhance therapeutic efficacy. This combination therapy can be personalized based on the patient's individual cardiovascular health and treatment response. The method may also involve routine blood tests to assess ketone levels and other biomarkers, allowing for adjustments in the dosage and composition of the ketone ester formulation to achieve optimal therapeutic results.

### Highly Purified (R)-3′-hydroxybutyl (R)-3′-hydroxybutyrate and Muscle Fatigue

In some embodiments, methods for reducing muscle fatigue may involve administering a highly purified (R)-3′-hydroxybutyl (R)-3′-hydroxybutyrate composition to a subject in the form of an orally ingested liquid solution. This liquid solution may be administered as a single dose or in multiple doses throughout the day, depending on the individual's needs and the extent and severity of muscle fatigue. In some embodiments, the highly purified (R)-3′-hydroxybutyl (R)-3′-hydroxybutyrate composition may be used in a solid form, such as a tablet or capsule, which may be taken with water. This form may be an option for subjects who struggle with liquid supplements or who require a more controlled release of the active ingredients. In some other examples, the highly purified (R)-3′-hydroxybutyl (R)-3′-hydroxybutyrate composition may be combined with other supplements known to support muscle function, such as branched-chain amino acids (BCAAs) or electrolytes, to enhance the effectiveness of the highly purified (R)-3′-hydroxybutyl (R)-3′-hydroxybutyrate composition. The method also includes measuring muscle endurance through various tests, such as isometric strength tests, endurance cycling, or running tests, to monitor the reduction in muscle fatigue over time. In a further example, the highly purified (R)-3′-hydroxybutyl (R)-3′-hydroxybutyrate composition may be administered in conjunction with a structured exercise program tailored to the subject's fitness level and goals, ensuring that the benefits of the ketone ester are enhanced through synergistic effects with physical training. The administration of the highly purified (R)-3′-hydroxybutyl (R)-3′-hydroxybutyrate composition may be adjusted based on the subject's response, with periodic assessments to optimize dosage and timing for optimal outcomes in reducing muscle fatigue.

### Highly Purified (R)-3′-hydroxybutyl (R)-3′-hydroxybutyrate and Taste Improvement

In some embodiments, the method for improving the taste of a highly purified (R)-3′-hydroxybutyl (R)-3′-hydroxybutyrate composition may involve formulating the composition with natural flavoring agents such as citrus extracts, mint, or berry flavors to mask the bitterness of the ketone ester. This approach may leverage the purified (R),(R)-form enantiomer's reduced bitterness compared to the S-form counterpart, enhancing the overall palatability of the composition. In another example, the highly purified (R)-3′-hydroxybutyl (R)-3′-hydroxybutyrate composition is combined with a sweetening agent like stevia or monk fruit extract, which not only improves the taste but also maintains the composition's low-calorie profile, making the composition suitable for health-conscious consumers. Additionally, the composition may be encapsulated in a gel capsule to minimize direct taste exposure, providing an alternative delivery method for those sensitive to the taste. Furthermore, the highly purified (R)-3′-hydroxybutyl (R)-3′-hydroxybutyrate composition may be integrated into a beverage formulation, such as a sports drink or a smoothie, where the flavors and textures of other ingredients help to further mask any residual bitterness, thereby enhancing the overall consumer experience. Each of these examples facilitates that the highly purified (R)-3′-hydroxybutyl (R)-3′-hydroxybutyrate composition may remain effective while significantly improving the taste, making the composition more appealing and easier to consume for a broader audience.

### General Summary of Clinical Relevance of Ingestion of Highly Purified (R)-3′-hydroxybutyl (R)-3′-hydroxybutyrate

As has been mentioned, there may be numerous benefits of ingesting highly purified compositions of (R)-3′-hydroxybutyl (R)-3′-hydroxybutyrate. In summary, these benefits may include metabolic benefits including but not limited to enhanced fat oxidation and weight loss, improved insulin sensitivity, increased metabolic flexibility, amelioration of metabolic syndrome, neurological benefits, neuroprotective effects against neurodegenerative diseases, improved cognitive function and memory. Other benefits may include treatment aspects for epilepsy and seizure disorders, improvements for mood and mood stabilization, cardiovascular benefits, reduced inflammation in cardiovascular tissues, improvement in lipid profiles, reduction in blood pressure, anti-inflammatory effects, systemic reduction of inflammatory markers, treatment for inflammatory disorders, improvements in energy production, improvements as an alternative energy source for brain and muscles, enhanced physical endurance and performance, improved cellular health, promotion of mitochondrial biogenesis, improvement in anti-aging effects at the cellular level, improvements in gut health, beneficial effects on gut microbiome composition, improvement in intestinal barrier function, improved anti-tumor effects in some cancer models, improvements in bone health, improvements in bone mineral density, improvements in the immune system, enhancement in immune system function, reduction in oxidative stress, antioxidant-like effects in various tissues, improvements in hormonal regulation, improved influence on hunger and satiety hormones, improved recovery from exercise, reductions in exercise-induced muscle damage, and improvements in respiratory function. Additional aspects may include benefits in respiratory conditions, improved protections for renal function, improved protective effects against kidney injury, improved liver health, reduction in liver fat accumulation, and improvements in reproductive health including benefits in some fertility issues. In some additional embodiments, additional aspects may include improvements in sleep quality, improvement in sleep patterns and quality, improved resilience to stress, enhancement of cellular stress resistance mechanisms, improvements in wound healing with acceleration of wound healing processes, and improvements in longevity with potential contribution to increased lifespan.

### Combinatorial Compositions with Highly Purified (R)-3′-hydroxybutyl (R)-3′-hydroxybutyrate or (R)-3-Hydroxybutyrate with Natural Supplements for Stress/Depression Treatment Protocols

As mentioned there may be numerous disease states or patient conditions where exogenous

supplementation with highly purified (R)-3′-hydroxybutyl (R)-3′-hydroxybutyrate or with (R)-3-Hydroxybutyrate may result in medical improvements. In some embodiments, these supplementation substances may be combined with other substances to create a composition for various treatment protocols. In some embodiments, combinatorial compositions including highly purified (R)-3′-hydroxybutyl (R)-3′-hydroxybutyrate or (R)-3-hydroxybutyrate may be useful in the treatment of stress responses. In some embodiments, metabolism of BHB in the prefrontal cortex may contribute to acute stress response, and other substances included in a combinatorial composition may enhance the effectiveness in these mechanisms or others.

In some embodiments, highly purified (R)-3′-hydroxybutyl (R)-3′-hydroxybutyrate or (R)-3-hydroxybutyrate may be combined with Gaba (Gamma-Aminobutyric Acid). Gaba may be helpful with a role as an inhibitory neurotransmitter in function of the central nervous system. For stress this may be particularly relevant in its neurological function with respect to the brain. In some embodiments, GABA incorporation into these compositions may benefit from a role GABA may play to reducing neuronal excitability by preventing nerve cells from over-firing, which in turn may help maintain balance in brain activity. A highly purified (R)-3′-hydroxybutyl (R)-3′-hydroxybutyrate or (R)-3-hydroxybutyrate combined with Gaba may create a composition that may support a treatment protocol for regulating anxiety, promoting relaxation and controlling muscle tone as non-limiting examples.

In some embodiments, highly purified (R)-3′-hydroxybutyl (R)-3′-hydroxybutyrate or (R)-3-hydroxybutyrate may be combined with 5-HTP (5-Hydroxytryptophan). Ingestion of compositions comprising 5-HTP may leverage its function as a precursor to serotonin production. Serotonin may have a role as a neurotransmitter with effects on sleep, appetite, and mood regulation. Although 5-HTP is produced within the body of a patient, exogenous supplementation may produce these improvements through the enhancement of serotonin production. A highly purified (R)-3′-hydroxybutyl (R)-3′-hydroxybutyrate or (R)-3-hydroxybutyrate combined with 5-HTP may create a composition that may support a treatment protocol for stress related improvement, mood enhancement, sleep regulation, reduction of migraine states as non-limiting examples. The combination may also have effects related to appetite suppression In some embodiments.

In some embodiments, highly purified (R)-3′-hydroxybutyl (R)-3′-hydroxybutyrate or (R)-3-hydroxybutyrate may be combined with L-theanine. L-theanine is an amino acid that may be found in various food substances. In some embodiments, ingestion of L-theanine may be helpful to stress conditions as well as other conditions. In some embodiments, ingestion of combinations of highly purified (R)-3′-hydroxybutyl (R)-3′-hydroxybutyrate or (R)-3-hydroxybutyrate may be combined with L-theanine to promote relaxation, enhance focus and support sleep. Exogenous ingestion of L-theanine may promote endogenous production of certain neurotransmitters, like gamma-aminobutyric acid (GABA), serotonin, and dopamine. A highly purified (R)-3′-hydroxybutyl (R)-3′-hydroxybutyrate or (R)-3-hydroxybutyrate combined with L-theanine may create a composition that may support a treatment protocol for stress related improvement, mood enhancement, and sleep regulation as non-limiting examples.

In some embodiments, highly purified (R)-3′-hydroxybutyl (R)-3′-hydroxybutyrate or (R)-3-hydroxybutyrate may be combined with Ashwagandha (Withania somnifera) for ameliorative effects in reduction of stress and anxiety. In some embodiments, the combination may result in a reduction in a cortisol level in a treated patient. The combination may enhance effects that its components may have for boosting energy in a user. In some embodiments, improvement of cognitive function may be aided by the ingestion of the combination. A highly purified (R)-3′-hydroxybutyl (R)-3′-hydroxybutyrate or (R)-3-hydroxybutyrate combined with L-theanine may create a composition that may support a treatment protocol for stress related improvement, mood enhancement, and sleep regulation as non-limiting examples.

In some embodiments, highly purified (R)-3′-hydroxybutyl (R)-3′-hydroxybutyrate or (R)-3-hydroxybutyrate may be combined with two plant extracts, Magnolia officinalis and Phellodendron amurense for ameliorative effects in reduction of stress and anxiety. In some embodiments, the combination may result in a reduction in a cortisol level in a treated patient. In some embodiments, the combination may work by influencing the body's hypothalamic-pituitary-adrenal (HPA) axis, which may play a role in stress response aspects. By helping to regulate the HPA Axis system, the combination may reduce a patient's production of cortisol in response to stress. In some embodiments, improvement of cognitive function may be aided by the ingestion of the combination. A highly purified (R)-3′-hydroxybutyl (R)-3′-hydroxybutyrate or (R)-3-hydroxybutyrate combined with Magnolia officinalis and Phellodendron amurense or Relora may create a composition that may support a treatment protocol for stress related improvement, mood enhancement, and sleep regulation as non-limiting examples.

In some embodiments, highly purified (R)-3′-hydroxybutyl (R)-3′-hydroxybutyrate or (R)-3-hydroxybutyrate may be combined with buspirone, which may be called Azaperone for ameliorative effects in reduction of stress and anxiety. In some embodiments, the combination may reduce anxiety by stimulating serotonin and dopamine receptors on nerves, thereby altering the chemical messages that nerves receive. A highly purified (R)-3′-hydroxybutyl (R)-3′-hydroxybutyrate or (R)-3-hydroxybutyrate combined with buspirone may create a composition that may support a treatment protocol for stress related improvement, mood enhancement, and sleep regulation as non-limiting examples.

In some embodiments, highly purified (R)-3′-hydroxybutyl (R)-3′-hydroxybutyrate or (R)-3-hydroxybutyrate may be combined with Rhodiola rosea for ameliorative effects in reduction of stress and anxiety. Rhodiola may include several bioactive compounds, including rosavins and salidroside. These constituents may have a role through an adaptogenic and stress-relieving mechanism. These compounds may help regulate the body's stress response and improve resilience to mental and physical challenges. In some embodiments, the combination of highly purified (R)-3′-hydroxybutyl (R)-3′-hydroxybutyrate or (R)-3-hydroxybutyrate with Rhodiola rosea may result in a reduction in a cortisol level in a treated patient. In some embodiments, the combination may work by influencing the body's hypothalamic-pituitary-adrenal (HPA) axis and reducing overactivity, which may play a role in stress response aspects. By helping to regulate the HPA Axis system, the combination may reduce a patient's production of cortisol in response to stress. In some embodiments, improvement of cognitive function may be aided by the ingestion of the combination. A highly purified (R)-3′-hydroxybutyl (R)-3′-hydroxybutyrate or (R)-3-hydroxybutyrate combined with Rhodiola rosea may create a composition that may support a treatment protocol for stress related improvement, mood enhancement, and sleep regulation as non-limiting examples.

In some embodiments, highly purified (R)-3′-hydroxybutyl (R)-3′-hydroxybutyrate or (R)-3-hydroxybutyrate may be combined with SAM-e (S-Adenosyl-L-methionine) for ameliorative effects in reduction of stress and anxiety. SAM-e may play a role in chemical reactivity in numerous processes in the body where it donates methyl groups to other molecules, such as DNA, proteins, lipids, and hormones. Supplementation with a combination including SAM-e may have a role in mood regulation through influence in the production of serotonin, dopamine, and norepinephrine neurotransmitters. These constituents may have a role through an adaptogenic and stress-relieving mechanism. These compounds may help regulate the body's stress response and improve resilience to mental and physical challenges. A highly purified (R)-3′-hydroxybutyl (R)-3′-hydroxybutyrate or (R)-3-hydroxybutyrate combined with SAM-e may create a composition that may support a treatment protocol for stress related improvement and mood enhancement as non-limiting examples.

In some embodiments, highly purified (R)-3′-hydroxybutyl (R)-3′-hydroxybutyrate or (R)-3-hydroxybutyrate may be combined with Vitamin D for ameliorative effects in reduction of stress and depression. It may have a significant role in seasonal affective disorder (SAD). It may play a role or an influence in the production of serotonin. Vitamin D may have anti-inflammatory properties, and since inflammation may have a role in depression processes the ingestion of Vitamin D may improve depression. A highly purified (R)-3′-hydroxybutyl (R)-3′-hydroxybutyrate or (R)-3-hydroxybutyrate combined with Vitamin D may create a composition that may support a treatment protocol for stress related improvement, mood enhancement as well as mitigation of depression as non-limiting examples.

In some embodiments, highly purified (R)-3′-hydroxybutyl (R)-3′-hydroxybutyrate or (R)-3-hydroxybutyrate may be combined with methyl folate for ameliorative effects in reduction of stress and depression. Methylfolate, also known as L-methylfolate or 5-MTHF (5-methyltetrahydrofolate), is a form of folate, a B-vitamin. Methylfolate may be involved in a number of cellular processes. It may be used by the body in several biochemical reactions, including DNA synthesis, repair, and methylation, as well as the production of neurotransmitters like serotonin, dopamine, and norepinephrine A highly purified (R)-3′-hydroxybutyl (R)-3′-hydroxybutyrate or (R)-3-hydroxybutyrate combined with methylfolate may create a composition that may support a treatment protocol for stress related improvement, mood enhancement as well as mitigation of depression as non-limiting examples.

In some embodiments, highly purified (R)-3′-hydroxybutyl (R)-3′-hydroxybutyrate or (R)-3-hydroxybutyrate may be combined with zinc for ameliorative effects in reduction of stress and depression. Zinc is generally important in brain function and accordingly also important in influencing mood regulation, depression and stress. Zinc may be involved in neurogenesis and associated processes. Zinc may affect neurotransmitters such as in a non-limiting perspective gamma-aminobutyric acid (GABA), which helps reduce anxiety. a number of cellular processes. Zinc may help regulate neurotransmitters such as glutamate as well. The balance between body levels of zinc and copper may also have a role in anxiety states, including DNA synthesis, repair, and methylation, as well as the production of neurotransmitters like serotonin, dopamine, and norepinephrine A highly purified (R)-3′-hydroxybutyl (R)-3′-hydroxybutyrate or (R)-3-hydroxybutyrate combined with zinc may create a composition that may support a treatment protocol for stress related improvement, mood enhancement as well as mitigation of depression as non-limiting examples.

In some embodiments, highly purified (R)-3′-hydroxybutyl (R)-3′-hydroxybutyrate or (R)-3-hydroxybutyrate may be combined with zinc for ameliorative effects in reduction of stress and depression. Zinc is generally important in brain function and accordingly also important in influencing mood regulation, depression and stress. Zinc may be involved in neurogenesis and associated processes. Zinc may affect neurotransmitters such as in a non-limiting perspective gamma-aminobutyric acid (GABA), which helps reduce anxiety. a number of cellular processes. Zinc may help regulate neurotransmitters such as glutamate as well. The balance between body levels of zinc and copper may also have a role in anxiety states, including DNA synthesis, repair, and methylation, as well as the production of neurotransmitters like serotonin, dopamine, and norepinephrine A highly purified (R)-3′-hydroxybutyl (R)-3′-hydroxybutyrate or (R)-3-hydroxybutyrate combined with zinc may create a composition that may support a treatment protocol for stress related improvement, mood enhancement as well as mitigation of depression as non-limiting examples.

In some embodiments, highly purified (R)-3′-hydroxybutyl (R)-3′-hydroxybutyrate or (R)-3-hydroxybutyrate may be combined with saffron for ameliorative effects in reduction of stress and depression. Saffron is a spice derived from the flower Crocus sativus. Saffron is a composition with numerous biologically active constituents, which may include in a non-limiting sense crocin, safranal, and picrocrocin. Crocin may have a role as an antioxidant. Safranal may have protective aspects for neural cells and processes. Picrocrocin may have a role in antidepressive and mood enhancing effect. All these processes may have a role in cognitive state and status. In some embodiments, saffron may increase the activity of neurotransmitters such as in a non-limiting sense GABA (gamma-aminobutyric acid), and such inhibitory neurotransmitters that may calm brain activity and accordingly may improve health through reduction in anxiety and stress. Saffron's anti-inflammatory properties may protect against effects of neuroinflammation, which may relate to mood disorders, anxiety, and neurodegenerative disease. A highly purified (R)-3′-hydroxybutyl (R)-3′-hydroxybutyrate or (R)-3-hydroxybutyrate combined with saffron may create a composition that may support a treatment protocol for stress-related improvement, mood enhancement as well as mitigation of depression as non-limiting examples.

In some embodiments, highly purified (R)-3′-hydroxybutyl (R)-3′-hydroxybutyrate or (R)-3-hydroxybutyrate may be combined with omega-3 for ameliorative effects in reduction of stress and depression. Omega-3 (or Omega-3 fatty acids) are essential polyunsaturated fats that may have a role in brain function and mental health. Omega-3 fatty acids are not produced in a human body, so they are obtained through diet or exogenous supplementation. Omega-3 compositions may include Eicosapentaenoic acid (EPA), Docosahexaenoic acid (DHA), and Alpha-linolenic acid (ALA) among others. These constituents may improve brain function in various ways. Since DHA is a critical component of neuronal cell membranes it has importance to structure of the brain. Levels of DHA may therefore be associated with better cognitive performance. EPA may influence neurotransmitter pathways, such as those involving serotonin and dopamine, which accordingly may result in improvements in mood regulation, conditions like depression and anxiety. Omega-3s may improve synaptic plasticity, which may improve signal transmission between neurons. This association may result in emotional regulation and improvements in cognitive function. In some embodiments, ingestion of omega-3s may influence the body's response to stress by lowering cortisol levels. Also, omega-3s may have anti-inflammatory properties, which may be linked to mood states and anxiety levels as well as generalized brain function. Omega-3's anti-inflammatory properties may protect against effects of neuroinflammation, which may relate to mood disorders, anxiety, and neurodegenerative disease. A highly purified (R)-3′-hydroxybutyl (R)-3′-hydroxybutyrate or (R)-3-hydroxybutyrate combined with omega-3 may create a composition that may support a treatment protocol for stress related improvement, mood enhancement, as well as mitigation of depression, as non-limiting examples.

In some embodiments, highly purified (R)-3′-hydroxybutyl (R)-3′-hydroxybutyrate or (R)-3-hydroxybutyrate may be combined with magnesium L-threonate for ameliorative effects in reduction of stress and depression. Magnesium L-threonate may have a unique characteristic in that it can cross the blood-brain barrier bringing a source of magnesium to the brain. In some embodiments, magnesium L-threonate may improve neuroplasticity with improved mood and cognitive aspects. It may also increase the activity of neurotransmitters such as in a non-limiting sense GABA (gamma-aminobutyric acid), and such inhibitory neurotransmitters that may calm brain activity and accordingly may improve health through reduction in anxiety and stress. Magnesium L-threonate may also reduce levels of cortisol with ingestion, with associated improvement in stress relief. Magnesium L-threonate's anti-inflammatory properties may protect against effects of neuroinflammation, which may relate to mood disorders, anxiety, and neurodegenerative disease. A highly purified (R)-3′-hydroxybutyl (R)-3′-hydroxybutyrate or (R)-3-hydroxybutyrate combined with magnesium L-threonate may create a composition that may support a treatment protocol for stress related improvement, mood enhancement as well as mitigation of depression as non-limiting examples.

In some embodiments, highly purified (R)-3′-hydroxybutyl (R)-3′-hydroxybutyrate or (R)-3-hydroxybutyrate may be combined with creatine for ameliorative effects in reduction of stress and depression. Creatine may have a unique characteristic in that it can enhance brain energy metabolism. Combination with supplementary ketones may provide energy sources for brain functions. Improvements in energy function may also improve cognitive status and states. It may also influence the activity of neurotransmitters such as in a non-limiting sense GABA (gamma-aminobutyric acid), and such inhibitory neurotransmitters that may calm brain activity and accordingly may improve health through reduction in anxiety and stress. Creatine may also reduce levels of cortisol with ingestion, with associated improvement in stress relief. Creatine 's anti-inflammatory properties may protect against effects of neuroinflammation, which may relate to mood disorders, anxiety, and neurodegenerative disease. A highly purified (R)-3′-hydroxybutyl (R)-3′-hydroxybutyrate or (R)-3-hydroxybutyrate combined with creatine may create a composition that may support a treatment protocol for stress related improvement, mood enhancement as well as mitigation of depression as non-limiting examples.

In some embodiments, highly purified (R)-3′-hydroxybutyl (R)-3′-hydroxybutyrate or (R)-3-hydroxybutyrate may be combined with various compounds for ameliorative effects in improvement of sleep quality. Combinations may include one or more of the following: Chloral Hydrate, which may also be known as Welldorm; Cloral Betaine; Melatonin, which may also be known as Circadin, Slenyto, and Syncrodin; Clomethiazole, which may also be known as Heminevrin; Diazepam, which may also be known as Diazemuls, Diazepam RecTubes, Stesolid, and Tensium; Diphenhydramine, which may also be known as Sominex; Lormetazepam, which may also be known as Dormagen; Pregabalin, which may also be known as Lecaent, and Lyrica; Loprazolam; Lorazepam; Nitrazepam, which may also be known as Mogadon; Oxazepam, which may also be known as Oxazepam; Promethazine, which may also be known as Phenergan, Sominex; Zolpidem, which may also be known as Stilnoct; Zopiclone, which may also be known as Zimovane; Alprazolam, which may also be known as Xanax; and Temazepam. A highly purified (R)-3′-hydroxybutyl (R)-3′-hydroxybutyrate or (R)-3-hydroxybutyrate combined with various compounds may create a composition that may support a treatment protocol for sleep improvement as non-limiting examples.

### Combinatorial Compositions with Highly Purified (R)-3′-hydroxybutyl (R)-3′-hydroxybutyrate or (R)-3-Hydroxybutyrate with Medicaments for Stress/Depression Treatment Protocols

In some embodiments, highly purified (R)-3′-hydroxybutyl (R)-3′-hydroxybutyrate or (R)-3-hydroxybutyrate may be combined with antidepressive pharmaceuticals for ameliorative effects in reduction of stress and depression. Antidepressants may include Selective serotonin reuptake inhibitors SSRIs, Serotonin-norepinephrine reuptake inhibitors (SNRIs), and Tricyclic antidepressant (TCAs) as non-limiting examples.

In some embodiments, highly purified (R)-3′-hydroxybutyl (R)-3′-hydroxybutyrate or (R)-3-hydroxybutyrate may be combined with antidepressive pharmaceuticals for ameliorative effects in reduction of stress and depression. Selective serotonin reuptake inhibitors may have unique characteristics by blocking the reuptake or reabsorption of serotonin into the presynaptic neuron. The result may increase the availability of serotonin in the synaptic cleft. A prolonged presence of serotonin may enhance neurotransmission and can improve mood and reduce anxiety. The combinations may include an enhancement to brain energy metabolism. Improvements in energy function may also improve cognitive status and states. It may also influence the activity of neurotransmitters such as in a non-limiting sense GABA (gamma-aminobutyric acid), and such inhibitory neurotransmitters that may calm brain activity and accordingly may improve health through reduction in anxiety and stress. Some examples of SSRIs for combination may include one or more of the following, in a non-limiting perspective, Fluoxetine, which may be known by the brand name Prozac, and having the chemical name N-methyl-3-phenyl-3-(4-trifluoromethylphenoxy)propylamine; Sertraline, which may be known by the brand name: Zoloft, and having the chemical name (S)-N, N-dimethyl-1-(3, 4-dichlorophenyl)-2-(1-piperazinyl)propan-1-amine; Paroxetine, which may be known by the brand name Paxil, and having the chemical name (3S, 4R)-3-(1-methyl-2-pyrrolidinyl)-4-(4-fluorophenyl)-1(2H)-naphthalenone; Citalopram, which may have the brand name: Celexa, and having the chemical name (S)-1-(3-dimethylaminopropyl)-1-(4-fluorophenyl)-1,3-dihydro-2H-benzimidazol-2-one; Escitalopram, which may be known by the brand name: Lexapro, and having the chemical name (S)-1-(3-dimethylaminopropyl)-1-(4-fluorophenyl)-1,3-dihydro-2H-benzimidazol-2-one (isomer of citalopram); and Fluvoxamine, which may be known by the brand name Luvox, and having the chemical name N-[3-(trifluoromethyl)phenyl]-(4)-oxo-4-phenyl-2-azetidinepropanamine. In combination with SSRIs an associated improvement in mood may result. A highly purified (R)-3′-hydroxybutyl (R)-3′-hydroxybutyrate or (R)-3-hydroxybutyrate combined with SSRIs may create a composition that may support a treatment protocol for stress related improvement, mood enhancement as well as mitigation of depression as non-limiting examples.

In some embodiments, highly purified (R)-3′-hydroxybutyl (R)-3′-hydroxybutyrate or (R)-3-hydroxybutyrate may be combined with antidepressive pharmaceuticals for ameliorative effects in reduction of stress and depression. Serotonin-norepinephrine reuptake inhibitors (SNRIs) may have unique characteristics by blocking the reuptake or reabsorption of serotonin and norepinephrine into the presynaptic neuron. The result may increase the availability of serotonin and norepinephrine in the synaptic cleft. A prolonged presence of serotonin and norepinephrine may enhance neurotransmission and can improve mood and reduce anxiety. The combinations may include an enhancement to brain energy metabolism. Improvements in energy function may also improve cognitive status and states. It may also influence the activity of neurotransmitters such as in a non-limiting sense GABA (gamma-aminobutyric acid), and such inhibitory neurotransmitters that may calm brain activity and accordingly may improve health through reduction in anxiety and stress. Some examples of SNRIs for combination may include one or more of the following, in a non-limiting perspective, Venlafaxine, which may have the brand names Effexor or Effexor XR; Desvenlafaxine, which may have the brand names Pristiq or Khedezla; Duloxetine, which may have the brand names Cymbalta, or Irenka; and Milnacipran, which may have the brand name Savella. In combination with SNRIs an associated improvement in mood may result. A highly purified (R)-3′-hydroxybutyl (R)-3′-hydroxybutyrate or (R)-3-hydroxybutyrate combined with SNRIs may create a composition that may support a treatment protocol for stress related improvement, mood enhancement as well as mitigation of depression as non-limiting examples.

In some embodiments, highly purified (R)-3′-hydroxybutyl (R)-3′-hydroxybutyrate or (R)-3-hydroxybutyrate may be combined with antidepressive pharmaceuticals for ameliorative effects in reduction of stress and depression. Tricyclic antidepressants (TCAs) may also have such characteristics by blocking the reuptake or reabsorption of serotonin and norepinephrine into the presynaptic neuron. The result may increase the availability of serotonin and norepinephrine in the synaptic cleft. A prolonged presence of serotonin and norepinephrine may enhance neurotransmission and can improve mood and reduce anxiety. The combinations may include an enhancement to brain energy metabolism. Improvements in energy function may also improve cognitive status and states. It may also influence the activity of neurotransmitters such as in a non-limiting sense GABA (gamma-aminobutyric acid), and such inhibitory neurotransmitters that may calm brain activity and accordingly may improve health through reduction in anxiety and stress. Some examples of TCAs for combination may include one or more of the following, in a non-limiting perspective, amitriptyline, which may be known by the brand name Elavil; imipramine, which may be known by the brand name Tofranil; nortriptyline, which may be known by the brand name Pamelor; doxepin, which may be known by the brand name Sinequan, clomipramine, which may be known by the brand name Anafranil, trimipramine, which may be known by the brand name Surmontil, and amoxapine, which may be known by the brand name Asendin. A highly purified (R)-3′-hydroxybutyl (R)-3′-hydroxybutyrate or (R)-3-hydroxybutyrate combined with TCAs may create a composition that may support a treatment protocol for stress related improvement, mood enhancement as well as mitigation of depression as non-limiting examples.

In some embodiments, highly purified (R)-3′-hydroxybutyl (R)-3′-hydroxybutyrate or (R)-3-hydroxybutyrate may be combined with benzodiazepine type pharmaceuticals for ameliorative effects in reduction of stress and depression. Benzodiazepines may also have such characteristics by increasing gamma-aminobutyric acid (GABA) activity. GABA is a chemical that nerve cells may use to communicate with each other. It also may reduce brain activity. It is believed that excessive activity in the brain may lead to anxiety or other psychiatric disorders, therefore combinations with benzodiazepines may reduce excessive neural activity that can lead to anxiety, making them effective for managing acute stress, panic attacks, and generalized anxiety disorder (GAD). The combinations may include an enhancement to brain energy metabolism. Improvements in energy function may also improve cognitive status and states. It may also influence the activity of neurotransmitters such as in a non-limiting sense GABA (gamma-aminobutyric acid) (in addition to the effects of Benzodiazepines,), and other such inhibitory neurotransmitters that may calm brain activity and accordingly may improve health through reduction in anxiety and stress. Some examples of Benzodiazepines for combination may include one or more of the following, in a non-limiting perspective, diazepam, which may be known by the brand name Valium, alprazolam, which may be known by the brand name Xanax; clonazepam, which may be known by the brand name Klonopin; lorazepam, which may be known by the brand name Ativan; chlordiazepoxide, which may be known by the brand name Librium, temazepam which may be known by the brand name Restoril, flurazepam, which may be known by the brand name Dalmane, triazolam, which may be known by the brand name Halcion, oxazepam, which may be known by the brand name Serax, midazolam, which may be known by the brand name Versed, clorazepate, which may be known by the brand name Traxene; and estazolam, which may be known by the brand name ProSom. A highly purified (R)-3′-hydroxybutyl (R)-3′-hydroxybutyrate or (R)-3-hydroxybutyrate combined with benzodiazepines may create a composition that may support a treatment protocol for stress-related improvement, mood enhancement as well as mitigation of depression as non-limiting examples.

In some embodiments, highly purified (R)-3′-hydroxybutyl (R)-3′-hydroxybutyrate or (R)-3-hydroxybutyrate may be combined with anticonvulsants modulate neurotransmitter activity in the brain, particularly by influencing the levels of gamma-aminobutyric acid (GABA), which as mentioned previously is an inhibitory neurotransmitter. They may also impact other neurotransmitters such as serotonin and glutamate, contributing to their mood-stabilizing effects. Anticonvulsant type pharmaceuticals may be useful for ameliorative effects in reduction of stress and depression. It is believed that excessive activity in the brain may lead to anxiety or other psychiatric disorders, therefore the combinations with anticonvulsants may reduce excessive neural activity that can lead to anxiety, making them effective for managing acute stress, panic attacks, and generalized anxiety disorder (GAD). The combinations may include an enhancement to brain energy metabolism. Improvements in energy function may also improve cognitive status and states. Some examples of anticonvulsants for combination may include one or more of the following, in a non-limiting perspective, divalproex sodium, which may be known by the brand name Depakote, carbamazepine, which may be known by the brand name Tegretol; lamotrigine, which may be known by the brand name Lamictal; gabapentin, which may be known by the brand name Neurontin; topiramate, which may be known by the brand name Topamax, oxcarbazepine, which may be known by the brand name Trileptal, vigabatrin, which may be known by the brand name Sabril, zonisamide, which may be known by the brand name Zonegran, and pregabalin, which may be known by the brand name Lyrica. More generally, antiseizure medications may be used in the combinations and may, in a non-limiting sense, include one or more of the following: levetiracetam, which may be known by the brand name Keppra; phenytoin, which may be known by the brand name Dilantin; phenobarbital, which may be known by the brand name Luminal; clobazam, which may be known by the brand name Onfi; ethosuximide, which may be known by the brand name Zarontin; felbamate, which may be known by the brand name Felbatol; lacosamide, which may be known by the brand name Vimpat; cenobamate, which may be known by the brand name Xcopri; eslicarbazepine acetate, which may be known by the brand name Aptiom; perampanel, which may be known by the brand name Fycompa; primidone, which may be known by the brand name Mysoline; acetazolamide, which may be known by the brand name Diamox; brivaracetam, which may be known by the brand name Briviact; valproic acid (or 2-Propenoic Acid, 2-Propyl-4-pentenoic Acid), which may also be known as Valproate; and rufinamide, which may be known by the brand name Banzel. A highly purified (R)-3′-hydroxybutyl (R)-3′-hydroxybutyrate or (R)-3-hydroxybutyrate combined with anticonvulsants or anti-seizure drugs may create a composition that may support a treatment protocol for stress related improvement, mood enhancement as well as mitigation of depression as non-limiting examples. In some protocols, a highly purified (R)-3′-hydroxybutyl (R)-3′-hydroxybutyrate or (R)-3-hydroxybutyrate combined with anti-seizure drugs while the user maintains a low carb diet.

In some embodiments, highly purified (R)-3′-hydroxybutyl (R)-3′-hydroxybutyrate or (R)-3-hydroxybutyrate may be combined with antihistamines to aid in sedation and restorative sleep. Hydroxyzine is an antihistamine with a brand name Atarax that causes sedation. It may help treat insomnia caused by anxiety, and other medical conditions. In some other examples other chemicals in the class of antihistamines may be useful in combination with highly purified (R)-3′-hydroxybutyl (R)-3′-hydroxybutyrate or (R)-3-hydroxybutyrate. The combinations may include an enhancement to brain energy metabolism. Improvements in energy function may also improve cognitive status and states. Some examples of other antihistamines for combination may include one or more of the following, in a non-limiting perspective, diphenhydramine, which may be known by the brand name Benadryl, chlorpheniramine, which may be known by the brand name Chlortrimeton; brompheniramine, which may be known by the brand name Dimetapp; hydroxyzine, which may be known by the brand name Atarax; clemastine, which may be known by the brand name Tavist, and promethazine, which may be known by the brand name Phenergan. A highly purified (R)-3′-hydroxybutyl (R)-3′-hydroxybutyrate or (R)-3-hydroxybutyrate combined with antihistamines may create a composition that may support a treatment protocol for stress related improvement, mood enhancement as well as mitigation of depression as non-limiting examples.

It is believed that excessive activity in the brain may lead to anxiety or other psychiatric disorders, therefore the combinations with anticonvulsants may reduce excessive neural activity that can lead to anxiety, making them effective for managing acute stress, panic attacks, and generalized anxiety disorder (GAD). The combinations may include an enhancement to brain energy metabolism. Improvements in energy function may also improve cognitive status and states. Some examples of anticonvulsants for combination may include one or more of the following, in a non-limiting perspective, divalproex sodium, which may be known by the brand name Depakote, carbamazepine, which may be known by the brand name Tegretol; lamotrigine, which may be known by the brand name Lamictal; gabapentin, which may be known by the brand name Neurontin; topiramate, which may be known by the brand name Topamax, oxcarbazepine, which may be known by the brand name Trileptal, vigabatrin, which may be known by the brand name Sabril, zonisamide, which may be known by the brand name Zonegran, and pregabalin, which may be known by the brand name Lyrica. A highly purified (R)-3′-hydroxybutyl (R)-3′-hydroxybutyrate or (R)-3-hydroxybutyrate combined with anticonvulsants may create a composition that may support a treatment protocol for stress-related improvement, and mood enhancement, as well as mitigation of depression, as non-limiting examples.

Generalized health as well as treatment of disease states related to stress, mood and depression may be improved by supporting mitochondrial function. A combination of highly purified (R)-3′-hydroxybutyl (R)-3′-hydroxybutyrate or (R)-3-hydroxybutyrate may be combined with supplements to support mitochondrial function and to aid in general health as well as treat disease states related to stress, mood and depression. Such mitochondrial function supporting supplements may include, in a non-limiting perspective, the following: Alpha-Lipoic Acid (Thioctic Acid), which may also be known as ALA; 3,5,4′-Trihydroxystilbene, which may also be known as Resveratrol; Arginine Alpha-Ketoglutarate, which may also be known as AAKG; Acetyl-L-Carnitine, which may also be known as Acetyl L-Carnitine; Epigallocatechin Gallate, which may also be known as EGCG; Quercetin-3-O-rutinoside, which may also be known as Rutin; Anthocyanosides (from Vaccinium myrtillus), which may also be known as Bilberry; (S)-2-Amino-3-(2-thioxo-1H-imidazol-4-yl)propanoic acid, which may also be known as Ergothioneine; Beta vulgaris, which may also be known as Beet Root; Ubiquinone, which may also be known as CoQ10; Mitoquinol Mesylate, which may also be known as MitoQ; 2-(3,4-Dihydroxyphenyl)ethanol, which may also be known as Hydroxytyrosol; Nicotinamide Mononucleotide, which may also be known as NMN; Buckminsterfullerene (C60) in Medium-Chain Triglycerides (MCT) oil, which may also be known as C60 MCT; 3,3′,4′,5,7-Pentahydroxyflavone, which may also be known as Quercetin; Riboflavin, which may also be known as Vitamin B2; L-Carnitine, which may also be known as Carnitine; and N-(Aminoiminomethyl)-N-methylglycine, which may also be known as Creatine. There may also be peptide combinations that act in similar manners to support mitochondrial function including the following: Elamipretide, which may also be known as SS-31; Mitochondrial Open Reading Frame of the 12S rRNA-c, which may also be known as MOTS-c; Forkhead Box O 4-DRI (FOXO4 D-Retro-Inverso peptide), which may also be known as FOXO4; and Humanin, which may also be known as Humanin Peptide. A highly purified (R)-3′-hydroxybutyl (R)-3′-hydroxybutyrate or (R)-3-hydroxybutyrate combined with mitochondrial supporting supplements or peptide combinations may create a composition that may support a treatment protocol for stress related improvement, mood enhancement as well as mitigation of depression as non-limiting examples.

In some embodiments, highly purified (R)-3′-hydroxybutyl (R)-3′-hydroxybutyrate or (R)-3-hydroxybutyrate may be combined with supplements to provide neuroprotective aspects. The combinations may include an enhancement to brain energy metabolism. Improvements in energy function may also improve cognitive status and states. Some examples of supplements for combination may include one or more of the following, in a non-limiting perspective, Propolis extract (from Apis mellifera), which may also be known as Green Propolis; diferuloylmethane, which may also be known as Curcumin; L-Glutathione, which may also be known as Glutathione; N-Acetylcysteine, which may also be known as NAC; N-Acetyl-5-methoxytryptamine, which may also be known as Melatonin; Polysaccharides (from Ganoderma lucidum), which may also be known as Reishi Mushroom; Ginsenosides (from Panax ginseng), which may also be known as RG3 Ginseng; Selenium (as selenomethionine or sodium selenite; Zinc (as zinc gluconate or zinc sulfate); L-Ascorbic Acid, which may also be known as Vitamin C; Eicosapentaenoic Acid (EPA) and Docosahexaenoic Acid (DHA), which may also be known as Omega-3; Phosphatidylcholine, Phosphatidylserine, and Phosphatidylethanolamine, which may also be known as Phospholipids; Isothiocyanates (from Brassica oleracea), which may also be known as Sulforaphane; 2-Aminoethanesulfonic Acid, which may also be known as Taurine; 2-(2-Oxopyrrolidino)acetamide, which may also be known as Piracetam; and Echinacoside and Acteoside (from Cistanche deserticola), which may also be known as Cistanche Deserticola; and various forms of B-vitamins (e.g., B1: Thiamine, B2: Riboflavin, B3: Niacin, B5: Pantothenic Acid, B6: Pyridoxine, B7: Biotin, B9: Folate, B12: Cobalamin). A highly purified (R)-3′-hydroxybutyl (R)-3′-hydroxybutyrate or (R)-3-hydroxybutyrate combined with supplements may create a composition that may support a neuroprotection.

In some embodiments, highly purified (R)-3′-hydroxybutyl (R)-3′-hydroxybutyrate or (R)-3-hydroxybutyrate may be combined with supplements to provide neuroactive aspects. The combinations may include an enhancement to brain energy metabolism. Improvements in energy function may also improve cognitive status and states. Some examples of supplements for combination may include one or more of the following, in a non-limiting perspective, 1,3,7-Trimethylxanthine, which may also be known as Caffeine; Theacrine, which may also be known as Teacrine; and 2-Oxo-1-pyrrolidine derivatives, which may also be known as Racetams (e.g., Piracetam, Aniracetam, Oxiracetam, etc.). A highly purified (R)-3′-hydroxybutyl (R)-3′-hydroxybutyrate or (R)-3-hydroxybutyrate combined with supplements may create a composition that may provide neuroactive aspects.

In some embodiments, highly purified (R)-3′-hydroxybutyl (R)-3′-hydroxybutyrate or (R)-3-hydroxybutyrate may be combined with supplements to support neuro-transmitting aspects. The combinations may include an enhancement to brain energy metabolism. Improvements in energy function may also improve cognitive status and states. Some examples of supplements for combination may include one or more of the following, in a non-limiting perspective, N-Acetylcysteine, which may also be known as NAC; L-Glutathione, which may also be known as Glutathione; Gamma-Aminobutyric Acid, which may also be known as GABA; N-Acetyl-L-Tyrosine, which may also be known as N-Acetyltyrosine; Alpha-Glycerylphosphorylcholine, which may also be known as Alpha GPC; 5-Hydroxytryptophan, which may also be known as 5-HTP; Magnesium L-Threonate, which may also be known as Mag Threonate; N-Acetyl-5-Methoxytryptamine, which may also be known as Melatonin; and Isothiocyanate derived from Glucoraphanin, which may also be known as Sulforaphane. A highly purified (R)-3′-hydroxybutyl (R)-3′-hydroxybutyrate or (R)-3-hydroxybutyrate combined with supplements may create a composition to support neuro-transmitting aspects.

### Blends Formed from Multiple Components with Highly Purified (R)-3′-hydroxybutyl (R)-3′-hydroxybutyrate or (R)-3-hydroxybutyrate

In some embodiments, highly purified (R)-3′-hydroxybutyl (R)-3′-hydroxybutyrate or (R)-3-hydroxybutyrate may be combined with various of the previous components to support brain function. The combinations may include an enhancement to brain energy metabolism. Improvements in energy function may also improve cognitive status and states. Some examples of components to include in such a blend may include many or all of the following, in a non-limiting perspective, bio-energetics; neurotrophic compounds, neurotransmitters, neuroplasticity supporting compounds, and nutritional fats. Bio-energetics may include: Ubiquinone, which may also be known as CoQ10; Nicotinamide Mononucleotide, which may also be known as NMN; Arginine Alpha-Ketoglutarate, which may also be known as AAKG; 3,5,4′-Trihydroxystilbene, which may also be known as Resveratrol/Trans-Stilbene; Epigallocatechin Gallate, which may also be known as EGCG; N-(Aminoiminomethyl)-N-methylglycine, which may also be known as Creatine; 1,3,7,9-Tetramethyluric Acid, which may also be known as Teacrine; 1,7-Dimethylxanthine, which may also be known as Paraxanthine; Acetyl-L-Carnitine, which may also be known as Acetyl-L-Carnitine; and Riboflavin, which may also be known as B2 (Vitamin B2). Neurotropic compounds may include Brain-Derived Neurotrophic Factor (BDNF) enhancers, which may also be known as BDNF enhancers; hericium erinaceus, which may also be known as Lion's Mane; and Green Propolis. Neurotransmitters may include Alpha-Glycerylphosphorylcholine, which may also be known as Alpha GPC, and N-Acetyl L-Tyrosine, which may also be known as N-Acetyl L-Tyrosine. Neuroplasticity enhancing compounds may include Magnesium, which may also be known as Mag; Zinc; Iron; and Acetyl-L-Carnitine. Nutritional fats may include Phosphatidylcholine, Phosphatidylserine, Phosphatidylethanolamine, which may also be known as Phospholipids; Eicosapentaenoic Acid (EPA) and Docosahexaenoic Acid (DHA), which may also be known as Omega-3; and Pentadecanoic Acid (C15:0), which may also be known as Fatty15. A highly purified (R)-3′-hydroxybutyl (R)-3′-hydroxybutyrate or (R)-3-hydroxybutyrate combined with various components as described in a blend to create a composition to support brain function.

### General Examples

Some examples describe a method for enhancing cognitive function by administering a combination of highly purified (R)-3′-hydroxybutyl (R)-3′-hydroxybutyrate and caffeine. This combination aims to improve mental alertness and focus by leveraging the cognitive-enhancing properties of caffeine alongside the metabolic benefits of the ketone ester.

Some examples focus on reducing anxiety through the administration of a combination of highly purified (R)-3′-hydroxybutyl (R)-3′-hydroxybutyrate and L-theanine. L-theanine may be known for its calming effects, and when combined with the ketone ester, it may help alleviate anxiety symptoms.

Some examples outline a method for improving mood by administering a combination of highly purified (R)-3′-hydroxybutyl (R)-3′-hydroxybutyrate and 5-HTP. 5-HTP is a precursor to serotonin, and this combination aims to enhance mood and emotional well-being.

Some examples describe a method for enhancing sleep quality by administering a combination of highly purified (R)-3′-hydroxybutyl (R)-3′-hydroxybutyrate and melatonin. Melatonin is a hormone that regulates sleep, and this combination seeks to improve sleep patterns and duration.

Some examples focus on supporting weight loss by administering a combination of highly purified (R)-3′-hydroxybutyl (R)-3′-hydroxybutyrate and green tea extract. Green tea extract may be known for its metabolism-boosting properties, and this combination aims to enhance fat oxidation and weight management.

Some examples outline a method for reducing inflammation by administering a combination of highly purified (R)-3′-hydroxybutyl (R)-3′-hydroxybutyrate and curcumin. Curcumin is an anti-inflammatory compound found in turmeric, and this combination seeks to alleviate inflammatory conditions.

Some examples describe a method for enhancing muscle recovery by administering a combination of highly purified (R)-3′-hydroxybutyl (R)-3′-hydroxybutyrate and branched-chain amino acids (BCAAs). BCAAs may be known to support muscle repair, and this combination aims to improve recovery post-exercise.

Some examples focus on improving insulin sensitivity by administering a combination of highly purified (R)-3′-hydroxybutyl (R)-3′-hydroxybutyrate and metformin. Metformin is a common anti-diabetic drug, and this combination seeks to enhance glucose regulation.

Some examples outline a method for supporting cardiovascular health by administering a combination of highly purified (R)-3′-hydroxybutyl (R)-3′-hydroxybutyrate and omega-3 fatty acids. Omega-3s may be known for their heart health benefits, and this combination aims to improve cardiovascular function.

Some examples describe a method for enhancing antioxidant defenses by administering a combination of highly purified (R)-3′-hydroxybutyl (R)-3′-hydroxybutyrate and vitamin C. Vitamin C is a powerful antioxidant, and this combination seeks to reduce oxidative stress.

Some examples focus on reducing stress by administering a combination of highly purified (R)-3′-hydroxybutyl (R)-3′-hydroxybutyrate and ashwagandha. Ashwagandha is an adaptogen known for its stress-relieving properties, and this combination aims to promote relaxation.

Some examples outline a method for improving joint health by administering a combination of highly purified (R)-3′-hydroxybutyl (R)-3′-hydroxybutyrate and glucosamine. Glucosamine may be known to support joint function, and this combination seeks to enhance joint flexibility and reduce discomfort.

Some examples describe a method for enhancing energy levels by administering a combination of highly purified (R)-3′-hydroxybutyl (R)-3′-hydroxybutyrate and Coenzyme Q10. CoQ10 is involved in energy production, and this combination aims to boost overall energy and vitality.

Some examples focus on supporting liver health by administering a combination of highly purified (R)-3′-hydroxybutyl (R)-3′-hydroxybutyrate and milk thistle extract. Milk thistle may be known for its liver-protective properties, and this combination seeks to enhance liver function.

Some examples outline a method for improving skin health by administering a combination of highly purified (R)-3′-hydroxybutyl (R)-3′-hydroxybutyrate and collagen peptides. Collagen supports skin elasticity, and this combination aims to enhance skin appearance and health.

Some examples describe a method for enhancing immune function by administering a combination of highly purified (R)-3′-hydroxybutyl (R)-3′-hydroxybutyrate and zinc. Zinc is essential for immune health, and this combination seeks to bolster the immune system.

Some examples focus on reducing symptoms of depression by administering a combination of highly purified (R)-3′-hydroxybutyl (R)-3′-hydroxybutyrate and SAM-e. SAM-e may be known for its mood-enhancing properties, and this combination aims to alleviate depressive symptoms.

Some examples outline a method for supporting bone health by administering a combination of highly purified (R)-3′-hydroxybutyl (R)-3′-hydroxybutyrate and calcium. Calcium facilitates bone strength, and this combination seeks to enhance bone density and health.

Some examples describe a method for enhancing focus and concentration by administering a combination of highly purified (R)-3′-hydroxybutyl (R)-3′-hydroxybutyrate and ginkgo biloba. Ginkgo biloba may be known for its cognitive benefits, and this combination aims to improve mental clarity.

Some examples focus on improving metabolic health by administering a combination of highly purified (R)-3′-hydroxybutyl (R)-3′-hydroxybutyrate and berberine. Berberine may be known for its metabolic benefits, and this combination seeks to enhance metabolic processes and health.

Some examples outline a method for enhancing cognitive resilience by administering a combination of highly purified (R)-3′-hydroxybutyl (R)-3′-hydroxybutyrate and Rhodiola rosea. Rhodiola is an adaptogen that supports mental resilience, and this combination aims to improve stress response.

Some examples describe a method for reducing oxidative stress by administering a combination of highly purified (R)-3′-hydroxybutyl (R)-3′-hydroxybutyrate and alpha-lipoic acid. Alpha-lipoic acid is a potent antioxidant, and this combination seeks to protect cells from oxidative damage.

Some examples focus on supporting adrenal health by administering a combination of highly purified (R)-3′-hydroxybutyl (R)-3′-hydroxybutyrate and licorice root extract. Licorice root may be known for its adrenal-supportive properties, and this combination aims to enhance adrenal function.

Some examples outline a method for improving gut microbiota by administering a combination of highly purified (R)-3′-hydroxybutyl (R)-3′-hydroxybutyrate and probiotics. Probiotics support gut health, and this combination seeks to enhance the balance of beneficial bacteria.

Some examples describe a method for enhancing mitochondrial function by administering a combination of highly purified (R)-3′-hydroxybutyl (R)-3′-hydroxybutyrate and PQQ (pyrroloquinoline quinone). PQQ supports mitochondrial health, and this combination aims to boost cellular energy production.

Some examples focus on reducing cholesterol levels by administering a combination of highly purified (R)-3′-hydroxybutyl (R)-3′-hydroxybutyrate and red yeast rice. A combination of berberine, citrus bergamot, niacin, and Red yeast rice may be known for its cholesterol-lowering effects, and this combination seeks to improve lipid profiles.

Some examples outline a method for supporting eye health by administering a combination of highly purified (R)-3′-hydroxybutyl (R)-3′-hydroxybutyrate and lutein. Lutein is essential for eye health, and this combination aims to protect vision and reduce eye strain.

Some examples describe a method for enhancing detoxification by administering a combination of highly purified (R)-3′-hydroxybutyl (R)-3′-hydroxybutyrate and chlorella. Chlorella may be known for its detoxifying properties, and this combination seeks to support the body's natural detox processes.

Some examples focus on improving hair health by administering a combination of highly purified (R)-3′-hydroxybutyl (R)-3′-hydroxybutyrate and biotin. Biotin supports hair growth and strength, and this combination aims to enhance hair appearance and health.

Some examples outline a method for reducing menopausal symptoms by administering a combination of highly purified (R)-3′-hydroxybutyl (R)-3′-hydroxybutyrate and black cohosh. Black cohosh may be known for alleviating menopausal symptoms, and this combination seeks to improve quality of life during menopause.

Some examples describe a method for supporting thyroid function by administering a combination of highly purified (R)-3′-hydroxybutyl (R)-3′-hydroxybutyrate and iodine. Iodine is essential for thyroid health, and this combination aims to enhance thyroid function and hormone balance.

Some examples focus on enhancing endurance by administering a combination of highly purified (R)-3′-hydroxybutyl (R)-3′-hydroxybutyrate and beetroot extract. Beetroot extract may be known for its endurance-boosting properties, and this combination seeks to improve physical performance.

Some examples outline a method for reducing blood pressure by administering a combination of highly purified (R)-3′-hydroxybutyl (R)-3′-hydroxybutyrate and hibiscus extract. Hibiscus extract may be known for its blood pressure-lowering effects, and this combination aims to support cardiovascular health.

Some examples describe a method for supporting joint flexibility by administering a combination of highly purified (R)-3′-hydroxybutyl (R)-3′-hydroxybutyrate and MSM (methylsulfonylmethane). MSM supports joint health, and this combination seeks to enhance joint mobility and reduce discomfort.

Some examples focus on improving cognitive performance by administering a combination of highly purified (R)-3′-hydroxybutyl (R)-3′-hydroxybutyrate and (Phospho lipids)phosphatidylserine, phosphatidylcholine/ethoamine. Phospholipids may be known for its cognitive benefits, and this combination aims to enhance mental acuity and memory.

Some examples outline a method for enhancing cellular repair by administering a combination of highly purified (R)-3′-hydroxybutyl (R)-3′-hydroxybutyrate and NAD+ /NMN/NR precursors. NAD+ precursors support cellular repair processes, and this combination seeks to promote cellular health and longevity.

Some examples describe a method for reducing allergy symptoms by administering a combination of highly purified (R)-3′-hydroxybutyl (R)-3′-hydroxybutyrate and quercetin. Quercetin may be known for its anti-allergy properties, and this combination aims to alleviate allergic reactions.

Some examples focus on supporting liver detoxification by administering a combination of highly purified (R)-3′-hydroxybutyl (R)-3′-hydroxybutyrate and dandelion root. Dandelion root may be known for its liver-supportive properties, and this combination seeks to enhance liver detoxification processes.

Some examples outline a method for enhancing muscle strength by administering a combination of highly purified (R)-3′-hydroxybutyl (R)-3′-hydroxybutyrate and creatine. Creatine may be known for its muscle-strengthening effects, and this combination aims to improve muscle power and performance.

Some examples describe a method for improving vascular health by administering a combination of highly purified (R)-3′-hydroxybutyl (R)-3′-hydroxybutyrate and grape seed extract. Grape seed extract may be known for its vascular benefits, and this combination seeks to enhance blood vessel function and circulation.

Some examples focus on reducing symptoms of arthritis by administering a combination of highly purified (R)-3′-hydroxybutyl (R)-3′-hydroxybutyrate and turmeric. Turmeric may be known for its anti-inflammatory properties, and this combination aims to alleviate arthritis symptoms and improve joint health.

Some examples outline a method for supporting cognitive health in aging by administering a combination of highly purified (R)-3′-hydroxybutyl (R)-3′-hydroxybutyrate and ginseng. Ginseng may be known for its cognitive benefits, and this combination seeks to support mental function in older adults.

Some examples describe a method for enhancing metabolic rate by administering a combination of highly purified (R)-3′-hydroxybutyl (R)-3′-hydroxybutyrate and cayenne pepper extract. Cayenne pepper extract may be known for its metabolism-boosting effects, and this combination aims to increase energy expenditure.

Some examples focus on reducing symptoms of PMS by administering a combination of highly purified (R)-3′-hydroxybutyl (R)-3′-hydroxybutyrate and evening primrose oil. Evening primrose oil may be known for alleviating PMS symptoms, and this combination seeks to improve menstrual health.

Some examples outline a method for supporting healthy aging by administering a combination of highly purified (R)-3′-hydroxybutyl (R)-3′-hydroxybutyrate and resveratrol. Resveratrol may be known for its anti-aging properties, and this combination aims to promote longevity and vitality.

Some examples describe a method for enhancing focus by administering a combination of highly purified (R)-3′-hydroxybutyl (R)-3′-hydroxybutyrate and bacopa monnieri. Bacopa monnieri may be known for its cognitive-enhancing effects, and this combination seeks to improve concentration and mental clarity.

Some examples focus on reducing symptoms of ADHD by administering a combination of highly purified (R)-3′-hydroxybutyl (R)-3′-hydroxybutyrate and magnesium. Magnesium may be known for its calming effects, and this combination aims to alleviate ADHD symptoms and improve focus.

Some examples outline a method for supporting skin hydration by administering a combination of highly purified (R)-3′-hydroxybutyl (R)-3′-hydroxybutyrate and hyaluronic acid. Hyaluronic acid may be known for its hydrating properties, and this combination seeks to enhance skin moisture and elasticity.

Some examples describe a method for enhancing athletic recovery by administering a combination of highly purified (R)-3′-hydroxybutyl (R)-3′-hydroxybutyrate and tart cherry extract. Tart cherry extract may be known for its recovery benefits, and this combination aims to reduce muscle soreness and improve recovery time.

Some examples focus on reducing symptoms of chronic fatigue by administering a combination of highly purified (R)-3′-hydroxybutyl (R)-3′-hydroxybutyrate and cordyceps. Cordyceps may be known for its energy-boosting properties, and this combination seeks to alleviate fatigue and improve energy levels.

Some examples outline a method for supporting cognitive clarity by administering a combination of highly purified (R)-3′-hydroxybutyl (R)-3′-hydroxybutyrate and acetyl-L-carnitine. Acetyl-L-carnitine may be known for its cognitive benefits, and this combination aims to enhance mental clarity and focus.

Some examples describe a method for enhancing immune resilience by administering a combination of highly purified (R)-3′-hydroxybutyl (R)-3′-hydroxybutyrate and echinacea. Echinacea may be known for its immune-supportive properties, and this combination seeks to bolster immune function and resilience.

Some examples focus on reducing symptoms of IBS by administering a combination of highly purified (R)-3′-hydroxybutyl (R)-3′-hydroxybutyrate and peppermint oil. Peppermint oil may be known for its digestive benefits, and this combination aims to alleviate IBS symptoms and improve gut health.

Some examples outline a method for supporting cognitive endurance by administering a combination of highly purified (R)-3′-hydroxybutyl (R)-3′-hydroxybutyrate and L-tyrosine. L-tyrosine may be known for its cognitive-enhancing effects, and this combination seeks to improve mental stamina and performance.

Some examples describe a method for enhancing cellular energy by administering a combination of highly purified (R)-3′-hydroxybutyl (R)-3′-hydroxybutyrate and ribose. Ribose may be known for its energy-boosting properties, and this combination aims to enhance cellular energy production and vitality.

Some examples focus on reducing symptoms of fibromyalgia by administering a combination of highly purified (R)-3′-hydroxybutyl (R)-3′-hydroxybutyrate and SAM-e. SAM-e may be known for its pain-relieving properties, and this combination seeks to alleviate fibromyalgia symptoms and improve quality of life.

Some examples outline a method for supporting cognitive agility by administering a combination of highly purified (R)-3′-hydroxybutyl (R)-3′-hydroxybutyrate and nootropic compounds. Nootropics may be known for their cognitive-enhancing effects, and this combination aims to improve mental flexibility and performance.

Some examples describe a method for enhancing metabolic flexibility by administering a combination of highly purified (R)-3′-hydroxybutyl (R)-3′-hydroxybutyrate and chromium picolinate. Chromium picolinate may be known for its metabolic benefits, and this combination seeks to improve the body's ability to switch between energy sources.

Some examples focus on reducing symptoms of eczema by administering a combination of highly purified (R)-3′-hydroxybutyl (R)-3′-hydroxybutyrate and primrose oil. Primrose oil may be known for its skin-soothing properties, and this combination aims to alleviate eczema symptoms and improve skin health.

Some examples outline a method for supporting cognitive vitality by administering a combination of highly purified (R)-3′-hydroxybutyl (R)-3′-hydroxybutyrate and lion's mane mushroom. Lion's mane may be known for its neuroprotective properties, and this combination seeks to enhance cognitive function and vitality.

A number of embodiments of the present disclosure have been described. While this specification contains many specific implementation details, there should not be construed as limitations on the scope of any disclosures or of what may be claimed, but rather as descriptions of features specific to particular embodiments of the present disclosure. While embodiments of the present disclosure are described herein by way of example using several illustrative drawings, those skilled in the art will recognize the present disclosure is not limited to the embodiments or drawings described. The drawings, and the detailed description thereto are not intended to limit the present disclosure to the form disclosed, but to the contrary, the present disclosure is to cover all modification, equivalents and alternatives falling within the spirit and scope of embodiments of the present disclosure as defined by the appended claims.

The headings used herein are for organizational purposes only and are not meant to be used to limit the scope of the description or the claims. As used throughout this application, the word “may” be used in a permissive sense (i.e., meaning having the potential to), rather than the mandatory sense (i.e., meaning must). Similarly, the words “include,” “including,” and “includes” mean including but not limited to. To facilitate understanding, reference numerals have been used, where possible, to designate like elements common to the figures.

The phrases “at least one,” “one or more,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C” and “A, B, and/or C” means A alone, B alone, C alone, A and B together, A and C together, B and C together, or A, B and C together.

The term “a” or “an” entity refers to one or more of that entity. As such, the terms “a” (or “an”), “one or more” and “at least one” can be used interchangeably herein. It is also to be noted the terms “comprising,” “including,” and “having” can be used interchangeably.

Certain features that are described in this specification in the context of separate embodiments can also be implemented in combination in a single embodiment. Conversely, various features that are described in the context of a single embodiment can also be implemented in combination in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while method steps may be depicted in the drawings in a particular order, this should not be understood as requiring that such operations be performed in the particular order shown or in a sequential order, or that all illustrated operations be performed, to achieve desirable results.

What is claimed is:

1. A method of treating an inflammatory disorder in a subject in need thereof, comprising:
administering to the subject a combination consisting of highly purified (R)-3′-hydroxybutyl (R)-3′-hydroxybutyrate; and
at least one nutraceutical selected from the group comprising: omega-3 fatty acids, curcumin extract, turmeric extract, ginger, Boswellia, bromelain, grape seed extract, resveratrol, quercetin, echinacea, milk thistle extract, chlorella, dandelion root, and beetroot extract;
thereby reducing one or more inflammatory biomarkers selected from interleukin-1β (IL-1β), interleukin-18 (IL-18), interleukin-6 (IL-6), tumor necrosis factor-alpha (TNF-α), and high-sensitivity C-reactive protein (hs-CRP), and
inhibiting NLRP3 inflammasome activation, or inhibiting NF-κB activity.

2. The method of claim 1, wherein the highly purified (R)-3′-hydroxybutyl (R)-3′-hydroxybutyrate has a stereochemical purity of at least 95% of an (R, R) enantiomer and is co-administered with the at least one nutraceutical.

3. The method of claim 1, wherein the highly purified (R)-3′-hydroxybutyl (R)-3′-hydroxybutyrate has a stereochemical purity of at least 99% of an (R, R) enantiomer and is co-administered with the at least one nutraceutical.

4. The method of claim 1, wherein the at least one nutraceutical comprises the omega-3 fatty acids and/or the curcumin or the turmeric extract.

5. The method of claim 1, wherein the at least one nutraceutical comprises one or more of the ginger, the Boswellia, and the bromelain.

6. The method of claim 1, wherein the at least one nutraceutical comprises the grape seed extract and/or the resveratrol and/or the quercetin.

7. The method of claim 1, wherein the inflammatory disorder is selected from rheumatoid arthritis, psoriatic arthritis, ankylosing spondylitis, psoriasis, atherosclerosis, Alzheimer's disease, inflammatory bowel disease including Crohn's disease and ulcerative colitis, asthma, chronic obstructive pulmonary disease, gout, non-alcoholic fatty liver disease, and non-alcoholic steatohepatitis.

8. The method of claim 1, further comprising monitoring the one or more inflammatory biomarkers using blood tests at baseline and at one or more time points during treatment.

9. A pharmaceutical composition comprising:
highly purified (R)-3′-hydroxybutyl (R)-3′-hydroxybutyrate; and
at least one nutraceutical selected from:
omega-3 fatty acids, curcumin or turmeric extract, ginger, Boswellia, bromelain, grape seed extract, resveratrol, quercetin, echinacea, milk thistle extract, chlorella, dandelion root, and beetroot extract; and
a pharmaceutically acceptable carrier;
wherein ingestion of the pharmaceutical composition by a subject reduces one or more inflammatory biomarkers in the subject selected from one or more of: IL-1β, IL-18, IL-6, TNF-α, hs-CRP, and inhibiting NLRP3 inflammasome activation, and inhibiting NF-κB activity.

10. The pharmaceutical composition of claim 9, wherein the highly purified (R)-3′-hydroxybutyl (R)-3′-hydroxybutyrate has a stereochemical purity of at least 95% of an (R,R) enantiomer.

11. The pharmaceutical composition of claim 9, wherein the highly purified (R)-3′-hydroxybutyl (R)-3′-hydroxybutyrate has a stereochemical purity of at least 99% of an (R,R) enantiomer.

12. The pharmaceutical composition of claim 9, wherein the at least one nutraceutical comprises the omega-3 fatty acids and/or the curcumin or the turmeric extract.

13. The pharmaceutical composition of claim 9, wherein the at least one nutraceutical comprises one or more of the ginger, the Boswellia, and the bromelain.

14. The pharmaceutical composition of claim 9, wherein the at least one nutraceutical comprises the grape seed extract and/or the resveratrol and/or the quercetin.

15. A kit comprising: a first container containing highly purified (R)-3′-hydroxybutyl (R)-3′-hydroxybutyrate; a second container containing at least one nutraceutical selected from omega-3 fatty acids, curcumin or turmeric extract, ginger, Boswellia, bromelain, grape seed extract, resveratrol, quercetin, echinacea, milk thistle extract, chlorella, dandelion root, and beetroot extract; and instructions for use for treating an inflammatory disorder by administering a combination and monitoring one or more inflammatory biomarkers selected from IL-1β, IL-18, IL-6, TNF-α, and hs-CRP.

16. The kit of claim 15, wherein the at least one nutraceutical comprises omega 3 fatty acids and/or curcumin or turmeric extract.

17. The kit of claim 15, wherein the instructions for use comprise directions to monitor the one or more inflammatory biomarkers at baseline and at one or more time points during treatment.

18. The kit of claim 15, wherein the instructions for use comprise directions to assess NLRP3 inflammasome activation as part of monitoring a subject's response to treatment.

19. The kit of claim 15, wherein the instructions for use comprise directions to assess NFκB activity as part of monitoring a subject's response to treatment.

20. The kit of claim 15, wherein the instructions for use comprise directions to assess reductions in IL 6 and hs CRP over a prespecified treatment interval.
